(19) United States
(12) Patent Application Publication
(10) Pub. No.: US 2013/0195918 A1
(43) Pub. Date: Aug. 1, 2013

(54) Equine Herpesvirus 1 Vaccine and Vector and Uses Thereof

(71) Applicants: Dennis J. O'Callaghan, Shreveport, LA (US); ByungChul Ahn, Shreveport, LA (US); Yunfei Zhang, Shreveport, LA (US); Nikolaus Osterrieder, Potsdam (DE)

(72) Inventors: Dennis J. O'Callaghan, Shreveport, LA (US); ByungChul Ahn, Shreveport, LA (US); Yunfei Zhang, Shreveport, LA (US); Nikolaus Osterrieder, Potsdam (DE)

(21) Appl. No.: 13/564,806

(22) Filed: Aug. 2, 2012

Related U.S. Application Data
(60) Provisional application No. 61/521,131, filed on Aug. 8, 2011.

(51) Int. Cl. C12N 7/00

(52) U.S. Cl. CPC C12N 7/00
USPC 424/229.1; 435/235.1; 424/93.2; 435/320.1

(57) ABSTRACT

We have constructed a mutant EHV-1 that is lacking the entire 12.7 kbp IR segment of the viral genome and found the mutant EHV-1 to be replication competent, to have the ability to replicate in mammalian cell types (including human cells), and to exhibit reduced virulence in the mouse model of EHV-1 virulence.

The benefit of the filing date of provisional U.S. application Ser. No. 61/521,131, filed 8 Aug. 2011, is claimed under 35 U.S.C. §119(e).

This invention was made with government support under grant number AI-22001 awarded by the National Institute of Allergy and Infectious Diseases and under grant number P20-RR018724 awarded by the National Center for Research Resources of the National Institutes of Health. The government has certain rights in the invention.

### TECHNICAL FIELD

This invention pertains to a new, mutant equine herpesvirus that has reduced virulence compared to the parent virus and replicates in a variety of mammalian cell types, and that can be used as a live vaccine virus to immunize equines against equine herpesvirus-1 infection or as a viral vector to introduce exogenous genes or antigens in equine and other mammalian species.

### BACKGROUND ART

Equine herpesvirus-1 (EHV-1) is a member of Genus Varicellovirus within the Alphaherpesvirinae subfamily. It is a major equine pathogen that causes severe diseases such as respiratory disease, epidemic abortion storms, and brain infections that lead to paralysis. Herpesvirus genomes are classified into groups A to F with regard to their structural properties, for example, the number and location of repeat and inverted sequences and the ability to exist in one, two, or four isomeric arrangements (Roizman, 1996; Roizman and Pellet, 2001). Herpesviruses, such as EHV-1 (Henry et al., 1981; O'Callaghan and Osterrieder, 2008; Whalley et al., 2007), with group D types of genomes have a fixed long region covalently linked to a short genomic region comprised of a pair of inverted repeat sequences that bracket the unique short segment.

Herpesviruses are currently being engineered for use as gene therapy vectors and for the development of recombinant vaccines (Rosas et al., 2008; Srinivasan et al., 2008; Yokoyama et al., 2008). Manipulation of the genome, such as the introduction or deletion of gene(s), can be carried out by homologous recombination utilizing full-length infectious genomes established as BACs in E. coli (Rudolph et al., 2002; Tischer et al., 2006). In herpesvirus genomes, the presence of repeat sequences makes manipulation of some genes difficult because deletion of a diploid gene may be rescued by the same gene that is located in the other repeat sequence (Ahn et al., 2010; Boldogkoi et al., 1998). This means that alterations of sequences within one repeat segment are repaired by homologous recombination events involving identical sequences within the other inverted repeat segment (Ahn et al., 2010; Boldogkoi et al., 1998). Therefore, for a potential vaccine, it would be preferable to manipulate viruses such that the viral genome presents a simpler structure and is less virulent, but it retains the ability to replicate and its other major biological properties.

The genomic sequence arrangement of EHV-1 (Henry et al., 1981; O'Callaghan and Osterrieder, 2008; Ruyechan et al., 1982; Whalley et al., 2007) is a group D type of genome and contains a short region with a central unique segment bracketed by a pair of inverted repeat sequences that allow the short region to invert relative to the long region. The group D type of genome of herpesviruses has sequences at one terminus that are repeated in an inverted orientation internally (Roizman, 1996; Roizman and Pellet, 2001). This type of structure is observed in the genomes of several members of the Alphaherpesvirinae subfamily, including human herpesvirus 3 (varicella-zoster virus), bovine herpesvirus 1, suid herpesvirus 1, gallid herpesvirus 1, equine herpesvirus 3, and equine herpesvirus 4 (Roizman, 1996; Roizman and Pellet, 2001).

Additionally, EHV-1 has a genome of 150,000 base pairs (bp) (Telford et al., 1992) and is comprised of a unique long (UL) region covalently linked to a short (S) region that is organized as a unique short segment (US) bracketed by a pair of identical internal repeat (IR) and terminal repeat (TR) sequences (Henry et al., 1981; Ruyechan et al., 1982; Whalley et al., 1981). Each inverted repeat sequence harbors six diploid genes (IR1 to IR6) and a portion of the Us1 (gene 68) gene.

The IR1 gene encodes a sole IE protein that governs early and some late gene expression and downregulates its own expression (Caughman et al, 1985; Harty, 1990; Smith et al., 1992; 1993). The early IR2 gene is located within the IE (IR1) ORF and generates the IR2 protein (IR2P) that strongly downregulates expression of all genes as a potent negative regulator (Kim et al., 2006). The IR3 gene, unique to EHV-1, is trans-activated by the IE protein (IEP), EICP0 protein (EICPOP) and IR4 protein (IR4P), and produces a non-coding 1 kb late transcript (Ahn et al., 2007; Holden et al., 1992a) that downregulates expression of the IE gene in a luciferase reporter system (Ahn et al., 2010). The early regulatory IR4P cooperates with the IEP to enhance expression of early and late viral genes (Holden et al., 1995) and comprises the major portion of the IR4/UL5 hybrid protein encoded by defective interfering particles (DIP) that can cause persistent EHV-1 infection (Chen et al., 1996, 1999; Ebner et al., 2008; Ebner and O'Callaghan, 2006). The IR5 gene encodes a late 236 amino acid protein that exhibits homology to the ORF64 protein of varicella-zoster virus and the Us10 protein of herpes simplex virus 1 (Holden et al., 1992b), the latter being a tegument phosphoprotein that co-purifies with the nuclear matrix (Yamada et al., 1997). The IR6 early gene, unique to EHV-1 and its close relative EHV-4, encodes a 33 kDa phosphoprotein that functions in nuclear egress and viral cell-to-cell spread (Breeden et al., 1992; O'Callaghan et al., 1994; Osterrieder et al., 1998), and it is a major determinant of virulence (Osterrieder et al., 1996b). Lastly, 631 bp of the 3′ end of the EHV-1 US1 ORF (a homolog of HSV-1 US2) extend into the IR, and the US1 and IR6 transcripts are 3′ co-terminal (Breeden et al., 1992).

U.S. Pat. No. 5,292,653 discloses a mutant equine herpesvirus type 1 that fails to produce any functional thymidine kinase and use of such mutants as vaccines and carriers for exogenous proteins.

U.S. Pat. No. 5,741,696 discloses recombinant equine herpesviruses using mutant equine herpesviruses with deletion of the DNA encoding the US2 gene and optionally further deletion or alteration of the DNA corresponding to one or more of the gpG, gpE, and TK genes.

U.S. Pat. No. 5,795,578 discloses the gene encoding the envelope glycoprotein of equine herpesvirus type 1, the glycoprotein D (gD) gene, and to antibodies against gD polypeptides.

U.S. Pat. Nos. 6,803,041 and 7,226,604 disclose a equine herpesvirus vaccine based on an inactivated EHV-1 (chemically inactivated EHV-1 KyA virus) and an adjuvant; and optionally includes antigens against other equine pathogens, such as inactivated EHV-4 and inactivated A1 and/or A2 strains of equine influenza virus.

U.S. Pat. No. 7,060,282 discloses equine herpesviruses (EHV) mutants involving changes to the immediate early gene of EHV.

### DISCLOSURE OF INVENTION

We have constructed a mutant EHV-1 that is lacking the entire 12.7 kbp IR segment of the viral genome (vL11ΔIR) and found the mutant EHV-1 to be replication competent, to have the ability to replicate in mammalian cell types (including human cells) tested in cell culture assays, and to exhibit reduced virulence in the mouse model of EHV-1 virulence. We have showed that the IR segment is dispensable for EHV-1 replication, but that the vL11ΔIR mutant exhibits a smaller plaque size and delayed growth kinetics. We also restored the IR to the mutant virus (vL11ΔIRR).

Western blot analyses of cells infected with the mutant vL11ΔIR showed that the synthesis of viral proteins encoded by the immediate-early, early, and late genes was reduced at immediate-early and early times, but by late stages of replication, reached wild type levels. Intranasal infection of CBA mice revealed that the vL11ΔIR was significantly reduced, as mice with the vL11ΔIR showed a decreased lung viral titer and a greater ability to survive infection compared to mice that were infected with either parental or revertant virus.

This new EHV-1 mutant is the first known generation of a group D herpesvirus that lacks an entire internal inverted repeat sequence, and its genome cannot undergo inversion of the short region. In addition, the mutant has only one copy of the six viral genes found in each inverted repeat sequence. Therefore, the mutant virus can be used to generate additional mutant viruses that lack one or more genes in the inverted repeat segments. Because the mutant EHV-1 virus has a large section deleted, it can accept exogenous genes and carry those genes into host mammalian cells. The mutant virus can thus act as a carrier for exogenous genes. The exogenous genes could be known “antigens” of certain infectious diseases, and therefore, the mutant EHV-1 with the antigen could be a vaccine against the antigen-derived disease.

Since the mutant virus replicates in a variety of cell types and has a genome of reduced size, it would be a potential vector in gene therapy to accept and express as much as 13,000 bp of foreign DNA (several genes). Also, since the mutant has reduced virulence as compared to the parent virus used to make the mutant, it itself can be used as a live vaccine virus to immunize equines in order to protect them against wild type EHV-1 infection.

In addition, the mutant EHV-1 virus can be used to study the effects of mutations in the six genes found in the remaining copy of the inverted repeat sequence. In the mutant EHV-1 there is only one copy of each of these six genes, while in the parent EHV-1 there are two copies of each of these genes. Thus, the mutant EHV-1 lacking the entire IR would simplify approaches to mutate or delete any of the six genes that map with the short region repeat sequence.

### BRIEF DESCRIPTION OF THE DRAWINGS

FIGS. 1A-1D show the EHV-1 RacL11 genomic structure and deletion of the 12.7 kbp IR segment as confirmed by PCR analysis. FIG. 1A shows the RacL11 EHV-1 genomic structure based on the DNA sequence of the Ab4 EHV-1. IR and TR are the internal repeat and terminal repeat segments, respectively. UL is the unique long region, and US is the unique short segment within the Short region. The entire IR within sequences 112934 bp to 125649 bp was removed by GalK positive selection followed by GalK counter selection. FIG. 1B shows PCR confirmation of the insertion of the GalK marker. PCR with primer sets specific to the GalK marker flanking sequences detected the predicted 1 kb (lane 1) and 2.4 kb (lane 3) fragments, respectively, from pL11ΔIR-GalK, but not from pRacL11 (M=size markers). FIG. 1C shows PCR confirmation of the removal of the GalK marker from pL11ΔIR-GalK. PCR with a primer set specific to IR flanking sequences detected the predicted 2.7 kb fragment (lane 2) from pL11ΔIR-GalK and the predicted 1.4 kb fragment (lane 3) from pL11ΔIR. FIG. 1D shows PCR confirmation of the restored IR in pL11ΔIRR. PCR with primer sets specific to IR and UL junction region or the IR and US junction region detected the predicted sizes of amplicons from pL11ΔIRR (lanes 1 and 3), but not from pL11ΔIR-GalK (lanes 2 and 4).

FIGS. 2A-2B show BamHI digestion patterns and Southern blot analysis to document construction of ΔIR EHV-1. FIG. 2A shows the results of BamHI digested pRacL11, pL11ΔIR-GalK, pL11ΔIR, and pL11ΔIRR DNAs separated on a 0.8% agarose gel. Black arrows indicate marker sizes (M=size markers). FIG. 2B shows Southern blot analysis of BamHI digested pRacL11, pL11ΔIR, and pL11ΔIRR DNAs separated on a 0.8% agarose gel and then transferred onto a membrane. The presence and absence of GalK marker in EHV-1 BAC DNAs were examined by Southern blot using a probe specific to the GalK marker.

FIGS. 3A-3B show plaque morphology and relative plaque size of parental RacL11 EHV-1, vL11ΔIR, and vL11ΔIRR. FIG. 3A shows representative plaque morphology in RK13 cells of parental virus, the IR-deleted vL11ΔIR, and the IR-restored vL11ΔIRR. FIG. 3B shows relative plaque size of the same cells as FIG. 3A. The plaque sizes were measured by using the ImageJ software program. Bars represent means of 60 plaques of each virus, and error bars represent standard deviations.

FIGS. 4A-4B show characterization of the vL11ΔIR genome and IE protein expression in cells infected with vL11ΔIRR. FIG. 4A shows confirmation of the absence of the GalK marker in the vL11ΔIR genome. PCR amplification with a primer set specific to IR flanking sequences was performed. Lanes 1, 2, 3, and 4 indicate the DNA templates of pL11ΔIR DNA, DNA of RK13 cells infected with vL11ΔIR, pRacL11 DNA, and DNA of RK13 cells infected with RacL11, respectively. FIG. 4B shows a comparison of the IEP expression in RK13 cells infected with RacL11 EHV-1 (lanes 3, 5, and 7) or vL11ΔIRR (lanes 4, 6, and 8). Detection of the IEP was performed. Lanes 1 and 2 indicate marker and mock-infected cells, respectively.

FIG. 5 shows tropism of vL11ΔIR in various cell types. Monolayer cultures of each of the given cell types were infected with vL11ΔIR or RacL11 EHV-1 at a multiplicity of infection (moi) of 1. After a 2 h virus attachment at 37° C., the infected cells were washed with PBS followed by adding equal volumes of growth medium. At 72 h post infection (hpi), samples were harvested and titered by plaque assay. Error bars indicate standard deviations.

FIGS. 6A-6C show the growth kinetics of parental, vL11ΔIR and vL11ΔIRR, and the results of quantitative real time PCR. RK13 cells were infected with the respective virus at a moi of 0.2, and intracellular and extracellular viruses were harvested at the indicated times post infection and then titered. FIG. 6A shows an intracellular viral titer. FIG. 6B shows an extracellular viral titer. FIG. 6C shows the results from quantitative real time PCR. RK13 cells were infected with RacL11, vL11ΔIR and vL11ΔIRR at a moi of 10 followed by incubation at 4° C. for 2 h and at 37° C. for 30 min, and then followed by PBS washing. Total DNAs were extracted from virus infected RK13 cells, and the relative number of viral genomes was quantified. Error bars indicate standard deviations. P values were p=0.54 for vL11ΔIR and RacL11, and p=0.56 for vL11ΔIR and vL11ΔIRR.

FIGS. 7A-7E show a comparison of the expression of viral immediate-early, early, and late proteins in RK13 cells infected with RacL11 EHV-1 or vL11ΔIR by Western blot analyses. For all FIGS. 7A-7E, the lanes are as follows: lane 1: protein markers; lane 2: mock-infected RK13 cells; lanes 3, 5, and 7: RacL11-infected RK13 cells; and lanes 4, 6, and 8: vL11ΔIR-infected RK13 cells. GAPDH was used to normalize protein loading. RK13 cells were infected with RacL11 EHV-1 or vL11ΔIR at a moi of 5, whole cell lysates were prepared, and then viral proteins were detected. FIG. 7A shows the detection of the immediate-early protein. FIG. 7B shows the detection of the early IR4 protein. FIG. 7C shows the detection of the early EICP0 protein. FIG. 7D shows the detection of the early UL5 protein. FIG. 7E shows the detection of the late glycoprotein D.

FIGS. 8A-8D show a percentage change in body weight and percent survival of mock-infected mice and mice infected with RacL11, vL11ΔIR, or vL11ΔIRR, and EHV-1 titers of mouse lungs. Mice were either intranasally inoculated with sterile medium as control, or they were infected with 1×10⁶ pfu of RacL11 EHV-1, vL11ΔIR, or vL11ΔIRR. The total virus was then isolated from the mouse lungs. Body weight was measured daily, and the Student's-t test was used to compare measurements of body weight between the groups. Error bars indicate standard deviations. FIG. 8A shows the percentage change in body weight of control CBA mice (n=5) or mice infected with RacL11 (n=9), vL11ΔIR (n=9), or vL (n=9). FIG. 8B shows a percent survival of mock infected mice (n=5), and mice infected with RacL11 EHV-1 (n=9), vL11ΔIR (n=9), or vL11ΔIRR (n=9). FIG. 8C shows viral titers from the lungs of live mice that were infected with RacL11 EHV-1 (n=3, black bars), vL11ΔIR (n=3, empty bars), or vL11ΔIRR (n=3, cross-hatched bars) at days 2, 3, and 4 post infection. FIG. 8D shows viral titers of the lungs from mice that had succumbed to infection with RacL11 (black bars), vL11ΔIR (empty bars), or vL11ΔIRR (cross-hatched bars). The number of mice that succumbed to infection at each day during 3 days post infection (dpi) to 5 dpi are n=4 (bar 1), n=5 (bar 2), n=1 (bar 3), n=4 (bar 4), n=1 (bar 5), and n=3 (bar 6).

### MODES FOR CARRYING OUT THE INVENTION

We have made a mutant equine herpesvirus 1 (EHV-1) lacking the entire 12,700 base pairs (BP) internal repeat (IR) segment of the viral genome and found the mutant EHV-1 to be replication competent, to have the ability to replicate in mammalian cell types (including human cells) tested in cell culture assays, and to exhibit reduced virulence in the mouse model of EHV-1 virulence.

Since the mutant EHV-1 virus replicates in a variety of cell types and has a genome of reduced size, it can be used as a vector in gene therapy to accept and express as much as 13,000 bp of foreign DNA (several genes). Also, since the mutant EHV-1 has reduced virulence as compared to the parent virus used to make the mutant, it itself can be used as a live vaccine virus to immunize equines to protect them against wild type EHV-1 infection. This new EHV-1 mutant is the first known generation of a group D herpesvirus that lacks an entire internal inverted repeat, and thus cannot undergo inversion of the SHORT region. Also, because the mutant has only one copy of the six viral genes found in each inverted repeat, it will readily allow generation of additional mutant viruses that lack one or more genes in the inverted repeat segments.

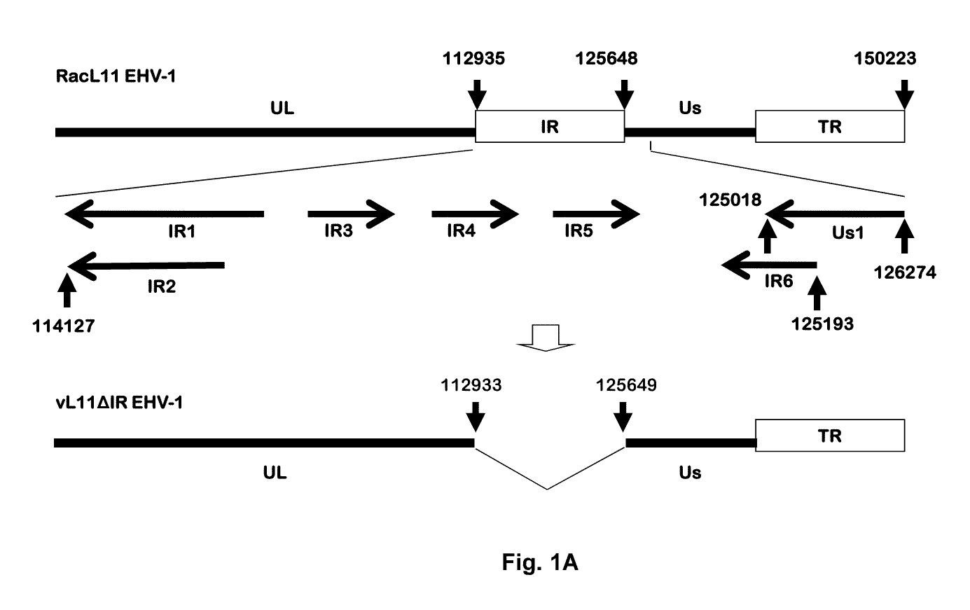

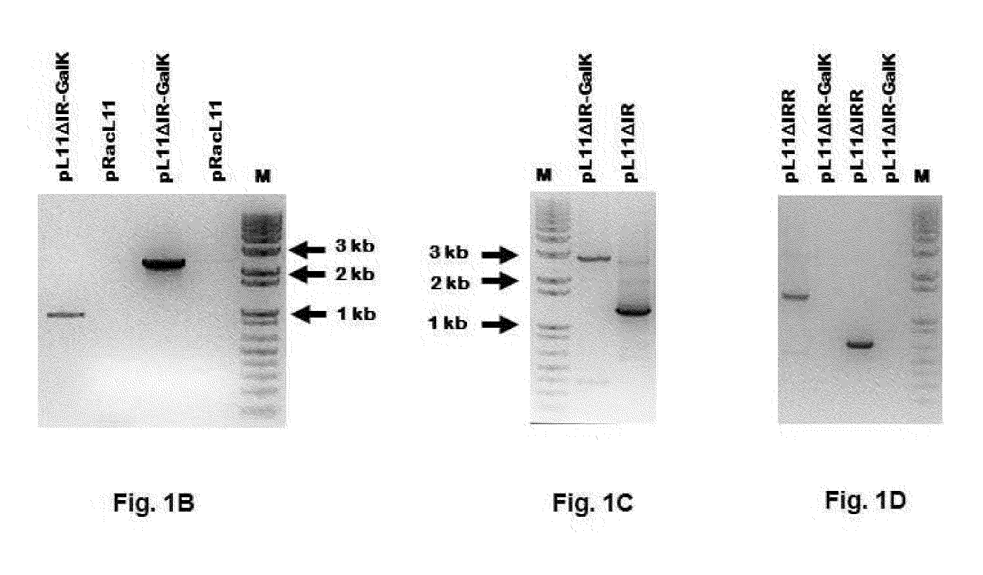

The diagram in FIG. 1A shows the EHV-1 RacL11 genomic structure and deletion of the 12.7 kbp IR segment. In FIGS. 1A, 1R and TR are the Internal Repeat and Terminal Repeat segments, respectively. UL is the Unique Long region, and Us is the Unique short segment within the Short region. As described below, the entire IR within sequences 112934 bp to 125648 bp was removed by GalK positive selection followed by GalK counter selection methods. The mutant EHV-1 virus (vL11ΔIR) with the deleted IR of 12,717 base pairs of DNA was shown to be capable of replication in mouse, rabbit, equine, monkey and human cell types.

We have created a mutant EHV-1 virus with a deletion of one of the two inverted repeat sequences. This virus thus has only one copy of the six genes found in the inverted repeat sequences—IR1, IR2, IR3, IR4, IR5, IR6, and a portion of US1 gene. A total of 12,715 base pairs were deleted as described below and as shown in FIG. 1A. We are not aware of another live EHV-1 mutant virus that could accept and express as much as 13,000 base pairs of foreign DNA (several genes). The apparent wide host range of this mutant will allow its use to introduce and express several genes in several different animal species.

The mutant EHV-1 virus with a deleted inverted repeat sequence was able to replicate in several types of mammalian cells, including mouse, equine, rabbit, monkey and human cells. The mutated virus was also shown to have lower virulence than the parental EHV-1 virus. Thus the virus can be used as a vaccine in horses to protect horses from EHV-1 infections. Equine herpesvirus type 1 is a major pathogen of equines worldwide with an enormous economic impact. EHV-1 causes respiratory symptoms through replication in epithelial cells of the upper respiratory tract, and causes fever, late-term abortions, and equine herpesvirus encephalomyelopathy (EHM or “equine stroke”). Thus the use of this mutant EHV-1 as a vaccine in horses should help prevent or decrease the symptoms associated with wildtype EHV-1 infections.

In addition, because the mutant EHV-1 virus has a large section deleted (over 12, 700 base pairs), it can accept exogenous genes and be used as a vector to deliver vaccine antigens and immunomodulatory genes into mammals. The mutant virus can thus act as viral vector to carry exogenous genes into mammals in which it replicates. We have shown the mutant EHV-1 has replicated in all five mammalian cells tested, cells from mice, horses, rabbits, monkeys, and human. The exogenous genes could be known “antigens” of certain infectious diseases, and thus the mutant EHV-1 with the antigen could be a vaccine against the antigen-derived disease. Examples of such exogenous genes that are antigens of other infectious diseases include, without limitation, genes expressing the Rabies G protein, equine infectious anemia ENV protein gp70-gp45, Eastern equine encephalitis virus E1 membrane protein, Eastern equine encephalitis virus E2 membrane protein, Venezuelan equine encephalitis virus E1 membrane protein, Venezuelan equine encephalitis virus E2 membrane protein, equine influenza virus hemagglutinin, equine influenza virus H3 protein, equine arteritis virus G1 membrane protein, equine arteritis virus G2 membrane protein, yellow fever virus prm-E protein, equine herpesvirus-4 glycoprotein gD, and other equine herpesviruses glycoproteins. The exogenous genes could also be known genes that encode proteins with known beneficial functions, e.g., proteins to increase or decrease the inflammatory response of the mammal. Examples include, without limitation, genes expressing interferon gamma (IFNγ), interleukin 12 (IL-12), and IL-2. To express the exogenous genes as proteins, these genes would need to be under the control of one or more promoters. Many such promoters are known in the literature, and examples, include without limitation, the EHV-1 immediate-early gene promoter, the EHV-1 tk gene promoter, the EHV-1 gp13 gene promoter, the EHV-1 gp 14 gene promoter, and the human cytomegalovirus immediate early gene promoter (See, for example, U.S. Pat. No. 5,292,653; and International Publication No. WO 2011/119925).

In addition, the mutant EHV-1 virus can be used to study the effects of mutations in the six genes found in the remaining copy of the inverted repeat sequence. In the mutant EHV-1 there is only one copy of each of these six genes, while in the parent EHV-1 there are two copies of each of these genes. The duplication of the six genes in the repeat segments of the short genomic region makes manipulation of these six genes quite problematic in the laboratory. Thus, the mutant EHV-1 lacking the entire IR would simplify approaches to mutate or delete any of the six genes that map with the short region repeat segment. For example, our previous work (Breitenbach, J. E., P. D. Ebner, and D. J. O'Callaghan 2009, Virology 383: 188-194) showed that the IR4 auxiliary regulatory protein is essential for EHV-1 pathogenesis and is a major factor in determining the host range of EHV-1. Thus, the delta-IR EHV-1 would be ideal to use as a parent virus to construct EHV-1 mutants with a deleted IR4 gene or with mutant forms of the IR4 gene; such IR4 mutants would be further attenuated and may have a limited tropism in the equine such as being incapable of replication in the lung or causing viremia that is an essential feature of the pathogenesis of outcomes such as abortion and infection of the central nervous system.

EHV-1 has been shown to exhibit a broad host range and replicates in a variety of cell types (O'Callaghan and Osterrieder, 2008; Trapp et al., 2005). Although closely related to EHV-1, EHV-4 has very limited cellular tropism that could be broadened when the EHV-4 gD gene was replaced with the EHV-1 homolog (Whalley et al., 2007). The fact that the tropism of vL11ΔIR was identical to that of the parental virus in the five cell types tested was interesting because recent studies with an EHV-1 mutant deleted of both copies of the IR4 gene showed a major change in its tropism as compared to that of the wild type EHV-1 (Breitenbach et al., 2009). Thus, a single copy of this auxiliary regulatory gene was sufficient for vL11ΔIR to replicate in the five cell types.

The virulence of EHV-1 in the CBA mouse model is well characterized by body weight loss and a significant mortality rate due to a massive inflammatory reaction in the lung mediated by the induction of cytokine/chemokine responses (Frampton et al., 2002; O'Callaghan and Osterrieder, 2008; Smith et al., 2005). We found that the vL11ΔIR was less virulent than the parental virus as judged by overall mortality and attribute this to the inability of this ΔIR mutant to replicate to high titers in the murine lung.

The term “vaccine” refers to a protein or any other biological agent, e.g., a virus with one or more antigens, in an administrable form capable of stimulating an immune response in a mammal given the vaccine and so confer resistance to the disease or infection in that mammal, including an ability of the immune system to remember the previously encountered antigen. For example, use of the mutant EHV-1 virus to stimulate an immune response in horses to confer resistance to equine herpesvirus-1 infection. Antibodies are produced as a result of the first exposure to an antigen and as a result of the initial immunization, a pool of memory B lymphocytes would be generated which could later produce antibodies. Thus in the event of subsequent exposure to the same antigen, the symptoms could be ameliorated, prevented, or decreased. In addition to the humoral immune response, the mutant EHV-1 virus would generate cell mediated immune responses (i.e., activation of T cells).

The term “adjuvant” refers to non-antigenic substance that, in combination with an antigen, enhances antibody production by inducing an inflammatory or other non-defined response, which leads to a local influx of antibody-forming cells. Adjuvants are used therapeutically in the preparation of vaccines, since they increase the production of antibodies against small quantities of antigen, lengthen the period of antibody production, and tend to induce memory cell responses. In the case of intranasal administration, the adjuvant may have bioadhesive properties to enhance exposure to the virus. Such adjuvants could include, but are not limited to, cross-linked polymers (e.g., as described in U.S. Pat. No. 6,803,041). Other adjuvants, particularly for administration by injection, include complete Freund's adjuvant, incomplete Freund's adjuvant, aluminum hydroxide, dimethyldioctadecylammonium bromide, Adjuvax (Alpha-Beta Technology), Imject Alum (Pierce), Monophosphoryl Lipid A (Ribi Immunochem Research), MPL+TDM (Ribi Immunochem Research), Titermax (CytRx), vitamin E acetate solubilisate, aluminum phosphate, aluminum oxide, toxins, toxoids, glycoproteins, lipids or oils, squalene, glycolipids, bacterial cell walls, subunits (bacterial or viral), carbohydrate moieties (mono-, di-, tri- tetra-, oligo- and polysaccharide) various liposome formulations or saponins Alum is the adjuvant currently in use for human patients. However, for horses, incomplete Freund's adjuvant may be used.

The term “immune response” refers to the reaction of the body to an antigen, which is usually a foreign or potentially dangerous substance (antigen), particularly disease-producing microorganisms. For example, in the current technology, the mutant EHV-1 virus would carry the antigen. The response involves the production by specialized white blood cells (lymphocytes) of proteins known as antibodies, which react with the antigens to render them harmless. The antibody-antigen reaction is highly specific. Vaccines such as the mutant EHV-1 also stimulate immune responses.

The term “immunologically effective amount” refers to the quantity of an immune response inducing substance required to induce the necessary immunological memory required for an effective vaccine. A vaccine is often given in multiple doses, an initial treatment and a subsequent booster treatment to enhance the immune response and to increase the strength and longevity of the immune memory response.

Typically, such vaccines are prepared to be administered in a sterile manner, either as liquid solutions or suspensions; solid forms suitable for solution in, or suspension in, liquid prior to injection may also be prepared. The preparation also may be emulsified. The active immunogenic ingredient is often mixed with an excipient that is pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH-buffering agents, adjuvants or immunopotentiators that enhance the effectiveness of the vaccine.

The vaccines are conventionally administered intraperitoneally, intramuscularly, intradermally, subcutaneously, orally, intranasally, or parenterally. Vaccines to be injected are typically formulated with pharmacologically acceptable carriers that are suitable for injection, including sterile aqueous solutions or dispersions. The carrier can be, for example, water, ethanol, glycerol, propylene glycol, sugars or other stabilizers, and isotonic saline solutions. Additional formulations are suitable for other modes of administration and include oral formulations. Oral formulations include such typical excipients as, for example, pharmaceutical grades of mannitol, lactose, starch, magnesium stearate, sodium saccharine, cellulose, magnesium carbonate and the like. Additionally, the peptide can be encapsulated in a sustained release formulations or a coating that resist the acidic pH of the stomach. The compositions take the form of solutions, suspensions, tablets, pills, capsules, sustained release formulations or powders and contain 10%-95% of active ingredient, preferably 25-70%.

The dose to be administered depends on a predetermined quantity of active material calculated to produce the desired therapeutic effect in association with the required diluent, i.e., carrier or vehicle, and a particular treatment regimen. The quantity to be administered, both according to number of treatments and amount, depends on the subject to be treated, capacity of the subject's immune system to synthesize antibodies, and degree of protection desired. The precise amounts of active ingredient required to be administered depend on the judgment of the practitioner and are peculiar to each individual. However, suitable dosage ranges are on the order of 10⁴ to 10⁷ PFU, more preferably 10⁵ to 10⁶ of active live virus per individual subject. Suitable regimes for initial administration and booster shots also vary but are typified by an initial administration followed in one or two week intervals by one or more subsequent injections or other administration. Annual boosters may be used for continued protection.

### Example 1

Materials and Methods

Cell culture and viruses. Mouse L-M, rabbit RK13, equine NBL-6, monkey Vero, and human HeLa cells used for viral propagation were maintained with Eagle's minimal medium supplemented with 100 units of penicillin/ml, 100 μg of streptomycin/ml, nonessential amino acids, and 5% (or 10%) fetal bovine serum. All cells were obtained from the American Type Culture Collection (ATCC, Manassas, Va.). All routine chemicals were obtained from Sigma-Aldrich (St. Louis, Mo.) or Fisher Scientific Company (Houston, Tex.). The pathogenic RacL11 EHV-1 strain (RacL11) (from Dr. Nikolaus Osterrieder) was used as the parental virus in our studies (Ahn et al., 2007; Ahn et al., 2010; Breitenbach et al., 2009).

Construction of Plasmids.

PCR products were amplified using Accuprime pfx polymerase (Invitrogen, Carlsbad, Calif.), pRacL11 EHV-1 BAC (pRacL11) template, and appropriate primers. GalK BAC technology was used in order to construct the IR-deleted EHV-1 (Ahn et al., 2010; Warming et al., 2005). pRacL11 (Rudolph et al., 2002) was transformed into SW106 E. coli (Warming et al., 2005). The purified PCR product of the GalK marker harboring the EHV-1 IR flanking sequences (Primers, 5′ ccg ggc cat atc tgg tca agg gtc acg ggc ccg cgc ccg aga gag agc ctg gcc cct gtt gac aat taa tca tcg gca 3′ (SEQ ID NO:1)/5′ aca ccg tag tgg gtg agt gtg ggt ttt cca aac ata gct cga att cat tag ttc agc act gtc ctg ctc ctt 3′ (SEQ ID NO:2)) was transfected into SW106 cells containing pRacL11. Positive colonies were selected on Gal positive selection agar plates (Warming et al., 2005), and confirmed by PCR amplification (left flanking region primers, 5′ atg atc ccg cag tta cag cct aca aac tgg 3′ (SEQ ID NO:3)/5′ tag cac acc taa cct cct gag tgt gag cg 3′ (SEQ ID NO:4); right flanking region primers, 5′ agt tga tgg ata ggc gag cat ctc aaa caa g 3′(SEQ ID NO:5)/5′ tga aac atc tgc aac tgc gta aca aca gct tcg g 3′ (SEQ ID NO:6)) of EHV-1 IR flanking regions (named pL11ΔIR-GalK). Counter selection was performed in order to remove the GalK marker from the intermediate (Ahn et al., 2010; Warming et al., 2005). Both flanking regions of the IR were combined by multiple rounds of PCR amplification (left flanking region primers, 5′ tag cac acc taa cct cct gag tgt gag cg 3′ (SEQ ID NO:4)/5′ aga tgt ata tct gcc agg ctc tct ctc ggg cg 3′(SEQ ID NO:7); right flanking region primers, 5′ aga tat aca tct act aat gaa ttc gag cta tgt ttg g 3′(SEQ ID NO:8)/5′ ttc tct ttg gat ggt ata aga caa tcg tcg 3′(SEQ ID NO:9); combined flanking region primers, 5′ tag cac acc taa cct cct gag tgt gag cg 3′(SEQ ID NO:4)/5′ ttc tct ttg gat ggt ata aga caa tcg tcg 3′(SEQ ID NO:9)).

Purified PCR amplification products of the IR flanking region were transfected into SW106 cells containing pL11ΔIR-GalK, and positive colonies were selected on the Gal counter selection plates (Ahn et al., 2010; Warming et al., 2005). To generate the revertant virus recovering the entire IR sequence, plasmid (pAYC177-XbaII/B1: harboring the entire IR sequence and IR flanking sequences of the EHV-1 genome) (Ahn et al., 2007) was electroplated into SW106 cells (from Dr. Lindsey Hutt-Fletcher, Louisiana State University Health Sciences Center, Shreveport, La.) containing pL11ΔIR-GalK (named pL11ΔIRR), and positive colonies were selected on the Gal counter selection plates (Ahn et al., 2010; Warming et al., 2005). The identity of the resulting final BAC clone, named pL11ΔIR and pL11ΔIRR, was confirmed by PCR targeting the flanking sequences of the IR-deleted BAC (primers, 5′ aca cat tga gtc ctt tct act ctc ctc ctc gg 3′ (SEQ ID NO:10)/5′ ttc tct ttg gat ggt ata aga caa tcg tcg 3′(SEQ ID NO:9)) and the flanking region of the revertant clone in which the IR had been restored (primers, 5′ ccg ttt gaa tgc gat tgg tgg g 3′(SEQ ID NO:11)/5′ gcg ttg tat cta gca gcc cac g 3′(SEQ ID NO:12) and 5′ aga gta ggc gtt cca tcc acg 3′(SEQ ID NO:13)/5′ gac cct acc aaa ggc gtg tag g 3′(SEQ ID NO:14)). The deletion and restoration of the entire IR was ultimately verified by sequence analysis of amplified PCR amplicons, BamHI digestion, and Southern blot analysis.

Generation of Recombinant EHV-1 from Cloned BAC DNA and DNA Isolation Form Virus-Infected RK13 Cells.

Purified pL11ΔIR DNA or pL11ΔIRR DNA and a plasmid DNA containing the EHV-1 US4 gene (gene 71) (Rudolph et al., 2002) were co-transfected into RK-13 cells by using the BD CalPhos Mammalian Transfection Kit (Clontech, Mountain View, Calif.) according to the manufacturer's directions. At three days post transfection (dpt), supernatants were harvested from DNA transfected RK13 cells, and virus reconstitution was examined by plaque assay. EHV-1 plaques lacking green fluorescence (suggesting replacement of the gene encoding green fluorescent protein (GFP) with the EUS4 sequence) were isolated by three rounds of plaque purification, and the resulting viruses were named vL11ΔIR or vL11ΔIRR.

Viruses were propagated in RK13 or NBL-6 cells (ATCC, Manassas, Va.), and titered according to standard procedures (Perdue et al., 1974). The deletion or restoration of the entire IR in the respective viruses was confirmed by the PCR amplification of the IR-flanking regions using virus-infected RK13 cell DNA as a template and primers (5′ ttc tct ttg gat ggt ata aga caa tcg tcg 3′ (SEQ ID NO:9), 5′ aca cat tga gtc ctt tct act ctc ctc ctc gg 3′(SEQ ID NO:10)). DNA from EHV-1-infected RK13 cells was prepared by using DNAzol reagent (Molecular Research Center, Inc., Cincinnati, Ohio) according to the manufacturer's instructions, and PCR was used as a template.

Southern and Western Blot Analyses.

To confirm the insertion of the GalK marker into pRacL11, the removal of the GalK marker from pL11ΔIR-GalK, and the replacement of the GalK marker from pL11ΔIR-GalK with the entire IR sequences, BamHI digested pRacL11, pL11ΔIR-GalK, pL11ΔIR, and pL11ΔIRR were separated on a 0.8% agarose gel and transferred onto a positively charged nylon membrane (Ambion, Austin, Tex.) by using a semi-dry electroblotter (Bio-Rad Laboratories, Hercules, Calif.). After DNA transfer, the membrane was placed on blot paper saturated with 0.5M NaOH for 15 min, briefly washed with 2× saline sodium citrate buffer (SSC), and incubated at 80° C. for 1 h. The PCR amplicon of the GalK marker (primers, 5′ cct gtt gac aat taa tca tcg gca tag 3′ (SEQ ID NO:15)/5′ act gtc ctg ctc ctt gtg atg g 3′ (SEQ ID NO:16)) was end-labeled with [γ-³²P]ATP (New England Nuclear Corporation, Boston, Mass.) and T4 polynucleotide kinase (Promega, Madison, Wis.) according to the manufacturer's directions. Radiolabeled probe was denatured by adding 1/10 volume of 3M NaOH, incubated for 10 min at room temperature, and then neutralized by adding an equal volume of 1M Tris-HCl (pH 7). Prehybridization, hybridization, and washing were performed using a NorthernMax Kit (Ambion, Austin, Tex.) followed by autoradiography using a phosphorimage screen and the molecular imager FX system (Bio-Rad Laboratories). For protein detection, RK13 cells were infected with parental RacL11 virus or vL11ΔIR at a multiplicity of infection (moi) of 5, and cells were harvested at 4, 6, and 12 hours post infection (hpi). Whole cell lysates of virus-infected cells were separated by dodecyl sulfate-polyacrylamide gel electrophoresis (SDS-PAGE), and then transferred to a nitrocellulose membrane (Ambion) by using a semi-dry electroblotter (Bio-Rad Laboratories). The IE, early (E; IR4, EICP0, UL5), and late (L; gD) proteins were detected by using monospecific rabbit polyclonal antibodies produced as previously described (Bowles et al., 1997; Caughman et al., 1995; Flowers and O'Callaghan, 1992; Holden et al., 1994; Zhao et al., 1995) as primary antibodies and anti-rabbit IgG[Fc]-alkaline phosphatase conjugate (Promega) as the secondary antibody. Proteins were visualized by incubating the membrane containing blotted proteins in an AP conjugate substrate (AP conjugate substrate kit, Bio-Rad Laboratories) according to the manufacturer's directions.

Plaque Morphology, Growth Kinetics, and Cell Tropism.

For the plaque assays, RK13 cell monolayers were infected with serial 10-fold dilutions of the respective viruses and overlaid with medium containing 1.5% methylcellulose at 2 hours after infection. At 4 days post infection (dpi), plaques were fixed with 10% formalin, stained with 0.5% crystal violet, and then counted (Perdue et al., 1974). Plaque sizes were measured by using the ImageJ software program (http://rebweb.nih.gov/ij/). For single step growth kinetics, RK13 cells in 25 mm flasks were infected at a moi of 0.2 with the respective viruses. After 1 h of viral attachment at 4° C., cells were washed with PBS, 4 ml of growth medium was added, and viruses were harvested at designated time points. To determine the intracellular viral titer, virus infected cells were washed with PBS followed by adding 4 ml of growth medium, and freeze and thaw cycle, and the virus was titered. To determine the extracellular viral titer, supernatants were used. To determine the cellular tropism, five cell types (L-M, RK13, NBL-6, Vero, and HeLa cells) were infected at a moi of 1 with mutant, revertant, or parental viruses. After virus attachment for 1 h at 4° C., the virus-infected cells were washed with PBS followed by adding normal growth medium, and then the total viral titers were examined at 3 dpi.

Quantitative Real Time (RT)-PCR.

To compare the number of viruses attached to the host cells, quantitative real time PCR assays were performed using the DNAs from virus infected cells as the template, the EHV-1 UL3 ORF region specific primer set (5′ ttt gaa ttc gcc acc atg ggg gcc tgc tgc tcc tct ag 3′(SEQ ID NO:17)/5′ tta tgt aca att cag acc gta tat ggt gtt ttg c 3′(SEQ ID NO:18)), rabbit GAPDH gene specific primer set (GeneBank:L2396.1; 5′ cat gtt tgt gat ggg cgt gaa cca 3′(SEQ ID NO:19)/5′ taa gca gtt ggt ggt gca gga t 3′(SEQ ID NO:20)), and iQ SYBR Green Supermix (Bio-Rad Laboratories) according to the manufacturer's directions. Confluent RK13 cells in the 6 well plates were infected with RacL11, vL11ΔIR, or vL at a moi of 10. Virus infected RK13 cells were incubated at 4° C. for 2 hours and then at 37° C. for 30 minutes followed by washing the virus infected RK13 cells with PBS. Total DNAs, including cellular and viral DNAs, were prepared from virus infected RK13 cells by using DNeasy Blood & Tissue kit (Qiagen Inc., Valencia, Calif.), and used as the template for quantitative RT-PCR. Cycle threshold (Ct) values to detect the viral genome were normalized by using Ct values of house-keeping GAPDH gene amplification.

Animal Experiments.

Animal experiments were also conducted using published procedures (Ahn et al., 2010; Frampton et al., 2002; Osterrieder et al., 1996b; von Einem et al., 2004). Groups of 4-week-old CBA female mice (Harlan Laboratories, Indianapolis, Ind.) were inoculated intranasally with sterile medium (mock infection) or 1×10⁶ pfu of vL11ΔIR, vL11ΔIRR or RacL11. Mice were observed daily and weighed from prior to inoculation, and the weights were compared. Virus isolation from the lungs of mice infected with vL11ΔIR, vL11ΔIRR, or RacL11 (n=3/group) at 2, 3, and 4 dpi for live mice and at the time of death for dead mice was performed by using silica beads and BeadBeater (BioSpec Products, Inc., Bartlesville, Okla.) according to the manufacturer's directions, and viral titers were then determined. For statistical analyses, two-tailed Student's-t test was performed by using the Excel software program (Microsoft Corporation, Redman, Wash.). Virulence as judged by percent survival data was determined by the Log-rank (Mantel-Cox) test using GraphPad Prism software (GraphPad Software, Inc., La Jolla, Calif.).

### Example 2

### The 12.7 Kbp IR Sequence of EHV-1 is Dispensable for Replication

We deleted the entire IR of the EHV-1 genome using GalK technology as previously described (Ahn et al., 2010; Rudolph et al., 2002; Warming et al., 2005), and characterized vL11ΔIR reconstituted from the recombinant BAC in cell culture. As shown in FIG. 1A, 12,715 bp of the EHV-1 genome that includes the entire IR and an additional 1 bp of the UL sequence were deleted.

The removal of the entire IR also resulted in deletion of 631 bp of the US1 gene (gene 68) that extends into the IR as shown in FIG. 1A (Breeden et al., 1992). Replacement of the entire IR with the GalK marker was confirmed by PCR amplification of two junction regions between the GalK marker and the EHV-1 genomic sequences at the UL terminus and the start of the US segment. FIG. 1B shows the PCR confirmation of the insertion of the GalK marker. PCR with primer sets specific to the GalK marker flanking sequences detected the predicted 1 kb (lane 1) and 2.4 kb (lane 3) fragments, respectively, from pL11ΔIR-GalK, but not from pRacL11 (M=size markers). FIG. 1C shows the PCR confirmation of the removal of the GalK marker from pL11ΔIR-GalK. PCR with a primer set specific to IR flanking sequences detected the predicted 2.7 kb fragment (lane 2) from pL11ΔIR-GalK and the predicted 1.4 kb fragment (lane 3) from pL11ΔIR. FIG. 1D shows the PCR confirmation of the restored IR in pL11ΔIRR. PCR with primer sets specific to IR and UL junction region or to the IR and US junction region detected the predicted sizes of amplicons from pL11ΔIRR (FIG. 1D, lanes 1 and 3), but not from pL11ΔIR-GalK (FIG. 1D, lanes 2 and 4).

PCR analyses indicated that the expected sizes of amplicons were observed from pL11ΔIR-GalK (FIG. 1B, lanes 1 and 3), but not from pRacL11 (FIG. 1B, lanes 2 and 4). Removal of the GalK marker from pL11ΔIR-GalK was confirmed by PCR amplification of the GalK marker flanking sequence (FIG. 1C, lanes 2 and 3) and DNA sequence analysis of PCR amplicons (data not shown). Replacement of the entire IR with the GalK marker from pL11ΔIR-GalK was confirmed by the PCR amplification of IR junction sequences and DNA sequence analysis (data not shown). Primer sets specific to UL (or US) and IR sequence amplified the expected sizes of PCR amplicons from pL11ΔIRR (FIG. 1D, lanes 1 and 3), but not from pL11ΔIR-GalK (FIG. 1D, lanes 2 and 4).

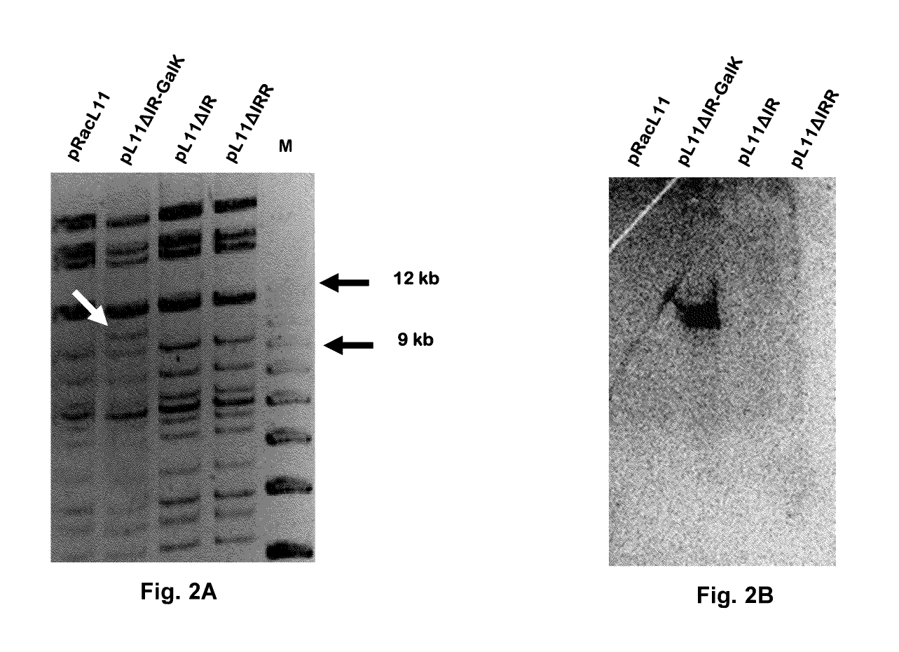

Deletion and recovery of the IR were further examined by BamHI digestion and Southern blot analyses. FIG. 2A shows the results of BamHI digested pRacL11, pL11ΔIR-GalK, pL11ΔIR, and pL11ΔIRR DNAs that were separated on a 0.8% agarose gel. Black arrows indicate marker sizes (M=size markers). FIG. 2B shows the results of Southern blot analysis for BamHI digested pRacL11, pL11ΔIR-GalK, pL11ΔIR, and pL11ΔIRR DNAs separated on a 0.8% agarose gel and then transferred onto a membrane. The presence and absence of GalK marker in EHV-1 BAC DNAs were examined by Southern blot using a probe specific to the GalK marker as described in Materials and Methods.

The BamHI digestion pattern showed that an additional band of approximately 10 kp in size was observed in the case of pL11ΔIR-GalK (FIG. 2A, lane 2), but pL11ΔIR lacking the 1.2 kb GalK marker showed an approximate 8.8 kb fragment instead of a 10 kb fragment (FIG. 2A, lane 3). The pL11ΔIRR showed a BamHI digestion pattern identical to that of pRacL11 (FIG. 2A, lanes 1 and 4).

To confirm that the pL11ΔIR-GalK harbored the GalK marker in the proper location, that pL11ΔIR lacks the GalK marker, and that the GalK marker from pL11ΔIR-GalK was replaced with the entire IR sequence, Southern blot analyses were performed using BamHI digested BAC DNAs (pRacL11, pL11ΔIR-GalK, pL11ΔIR, and pL11ΔIRR) and a radiolabeled GalK marker PCR fragment as the probe. These analyses showed that the GalK marker probe bound only to one fragment of the BamHI digested pL11ΔIR-GalK DNA (FIG. 2B, lane 2), but not to any band of pRacL11 used as the control (FIG. 2B, lane 1), pL11ΔIR (FIG. 2B, lane 3), or pL11ΔIRR (FIG. 2B, lane 4), indicating that the entire IR of the pRacL11 was correctly replaced with the GalK marker, that the GalK marker was removed in the final pL11ΔIR, and that the entire IR was restored in pL11ΔIRR.

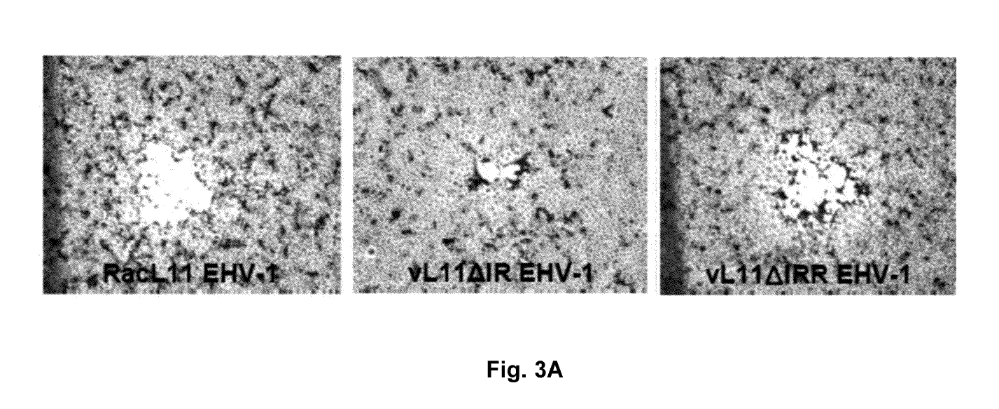

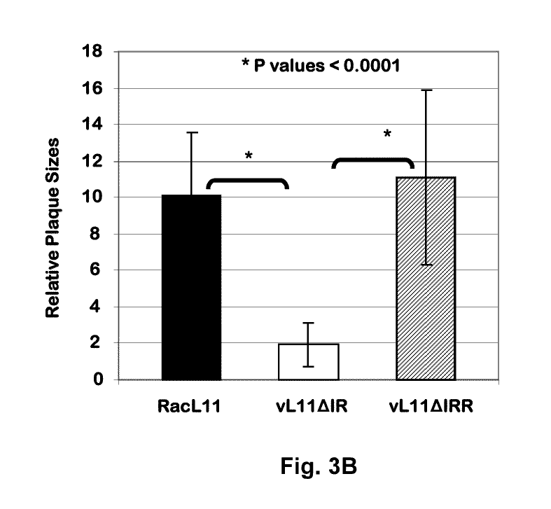

Once the deletion and restoration of the entire IR were confirmed, the recombinant vL11ΔIR and vL11ΔIRR viruses were generated by co-transfection of pL11ΔIR (or pL11ΔIRR) DNA and a plasmid containing the EHV-1 US4 gene (gene 71) produced as previously described (Rudolph et al., 2002). FIGS. 3A and 3B show the plaque morphology and relative plaque size of parental RacL11 EHV-1, vL11ΔIR, and vL11ΔIRR. FIG. 3A shows the representative plaque morphology in RK13 cells of parental virus, the IR-deleted vL11ΔIR, and the IR-restored vL11ΔIRR. FIG. 3B shows the relative plaque size. The plaque sizes were measured by using the ImageJ software program (http://rebweb.nih.gov/ij/). Bars represent means of 60 plaques of each virus; error bars represent standard deviations.

Successful reconstitution of vL11ΔIR cloned DNA indicated that the IR deletion virus was replication competent, but plaque assays showed that the plaque areas of vL11ΔIR were significantly reduced compared to those of parental RacL11 and vL11ΔIRR (p<0.0001; FIGS. 3A and B).

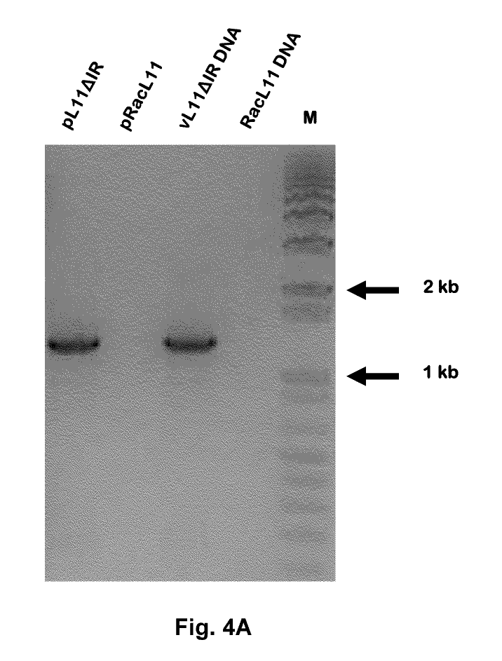

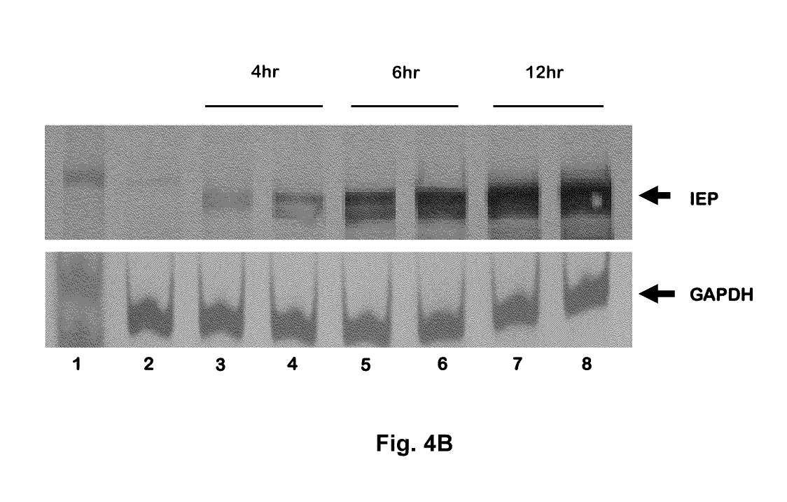

To exclude the possibility that the entire IR was restored by the TR segment during serial virus passage in RK13 cells, the IR flanking region of the vL11ΔIR genome was PCR-amplified by a primer set specific for the IR flanking sequences. Characterization of the vL11ΔIR genome and IE protein expression in cells infected with vL11ΔIRR is shown in FIGS. 4A and 4B. FIG. 4A shows the confirmation of the absence of the GalK marker in the vL11ΔIR genome. PCR amplification with a primer set specific to IR flanking sequences was performed as described in Example 1. Lane 1, 2, 3, and 4 indicate the DNA templates of pL11ΔIR DNA, DNA of RK13 cells infected with vL11ΔIR, pRacL11 DNA, and DNA of RK13 cells infected with RacL11, respectively. PCR amplicons of the same size were generated from both pL11ΔIR (FIG. 4A, lane 1) and DNA derived from vL11ΔIR-infected RK13 cells (FIG. 4A, lane 3). However, no amplicon was detected in DNA prepared from pRacL11 (FIG. 4A, lane 2) or from RacL11-infected RK13 cell DNA (FIG. 4A, lane 4), indicating that the IR segment was not repaired by recombination events with TR sequences during vL11ΔIR replication in RK12 cells.

To address whether the IR sequences restored in the revertant virus were functionally similar to the parental virus with respect to gene expression, synthesis of the IEP was examined in the various viruses. FIG. 4B shows the comparison of the IEP expression in RK13 cells infected with RacL11 EHV-1 (lanes 3, 5, and 7) or vL11ΔIRR (lanes 4, 6, and 8). Detection of the IEP was performed as described in Example 1. Lanes 1 and 2 indicate marker and mock-infected cells, respectively. IEP expression levels of both parental RacL11 and vL11ΔIRR viruses were similar at immediate-early, early, and late times of replication (FIG. 4B), results that indicated that the IR was completely restored in vL11ΔIRR.

### Example 3

### Cellular Tropism and Growth Kinetics of vL11ΔIR

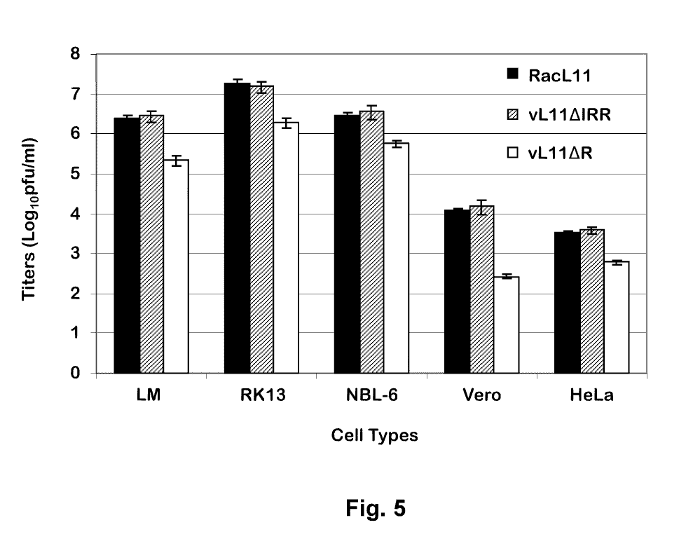

Even though the IR was not essential for EHV-1 replication, there remained the possibility that the cellular tropism of vL11ΔIR may differ from that of the parental virus. Recent studies had revealed that a mutant EHV-1 in which both copies of the IR4 gene were absent was capable of replication in equine NBL-6 cells, but, unlike its parent virus, was not capable of replication in mouse, rabbit, monkey, or human cells (Breitenbach et al., 2009). These observations suggested that the deletion of the entire IR may affect the biological properties of EHV-1. FIG. 5 shows the results of tropism of vL11ΔIR in five cell types: Mouse L-M, rabbit RK13, equine NBL-6, monkey Vero, and human HeLa cells. Monolayer cultures of each of the five cell types were infected with vL11ΔIR, vL11ΔIRR, or RacL11 EHV-1 at a moi of 1. After a 2 h virus attachment at 37° C., the infected cells were washed with PBS followed by adding equal volumes of growth medium. At 72 hpi, samples were harvested and titered by plaque assay as described in Materials and Methods. Error bars indicate standard deviations.

Investigation of the cellular tropism and replication of EHV-1 showed that vL11ΔIR, like the parental RacL11, was capable of replicating in all five cell types tested, but the vL11ΔIR replicated with significantly reduced titers when compared with the parental virus and the revertant virus (vL11ΔIRR) in all cell types examined (all p values were <0.05; FIG. 5). That the tropism of vL11ΔIR was identical to that of the parental virus in the five cell types tested was interesting because recent studies with an EHV-1 mutant deleted of both copies of the IR4 gene showed a major change in its tropism as compared to that of the wt EHV-1 (Breitenbach et al., 2009). Thus, a single copy of this auxiliary regulatory gene was sufficient for vL11ΔIR to replicate in the five cell types

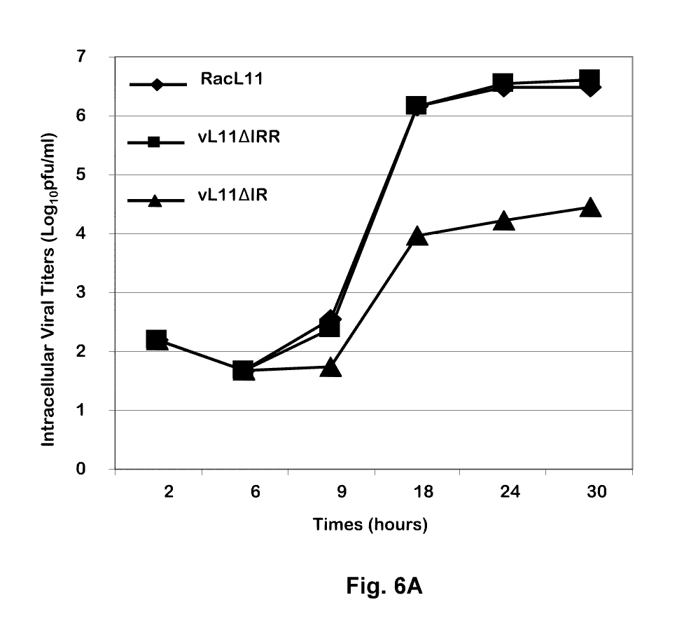

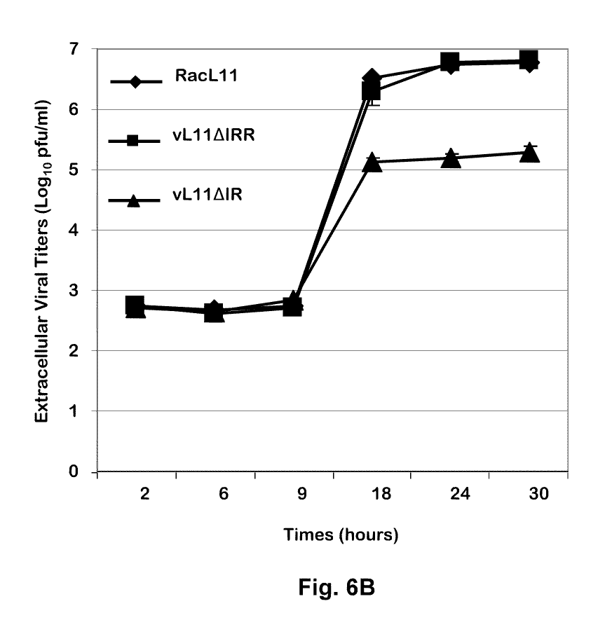

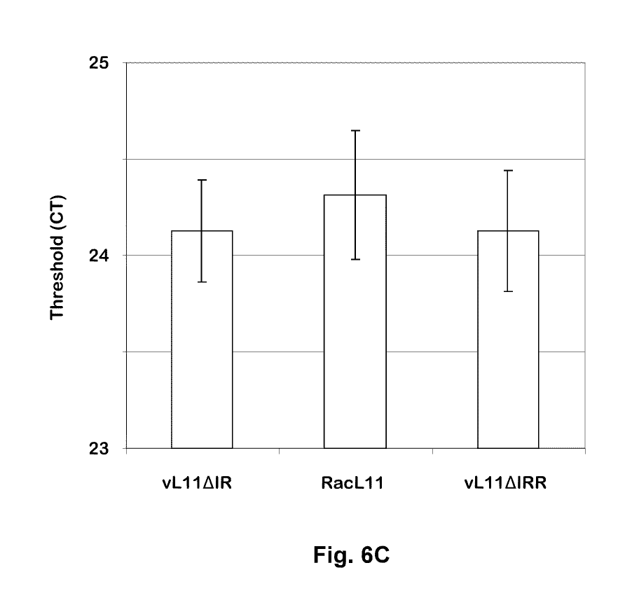

The growth kinetics of vL11ΔIR was analyzed in RK13 cells by examining intracellular and extracellular viral titers at various times after infection. The results are shown in FIGS. 6A and 6B. RK13 cells were infected with the respective virus at a moi of 0.2, and intracellular and extracellular viruses were harvested at the indicated times post infection and titered as described in Example 1. FIG. 6A shows the intracellular viral titer, and FIG. 6B shows the extracellular viral titer. Overall, growth of the vL11ΔIR was impaired as compared to that of the RacL11 as its replication exhibited a lag in reaching maximal titer. Both viruses reached maximal titers at 18 to 24 hours post infection, but the titer of the RacL11 and vL11ΔIRR exceeded that of the vL11ΔIR by more than one log.

To examine whether the delayed growth of vL11ΔIR was due to an impaired ability of the mutant virus in entry/penetration, cell-associated viral DNA was quantified by real time PCR after the parental virus, vL11ΔIR, and the revertant virus were incubated with RK cells. RK13 cells were infected with RacL11, vL11ΔIR and vL11ΔIRR at a moi of 10 followed by incubation at 4° C. for 2 h and at 37° C. for 30 min, and PBS washing. Total DNAs were extracted from virus infected RK13 cells, and the relative number of viral genomes was quantified as described in Example 1. The results are shown in FIG. 6C, where the error bars indicate standard deviations. P values for FIG. 6C were P=0.54 for vL11ΔIR and RacL11 and P=0.56 for vL11ΔIR and vL11ΔIRR. Comparison of Ct values of the RacL11, vL11ΔIR, and vL11ΔIRR DNAs revealed no significant difference (FIG. 6C). All p values were greater than p=0.50, suggesting that the delayed growth of vL11ΔIR is due to a reduced rate of replication rather than impaired virus entry/penetration.

Deletion of the EHV-1˜13 kbp IR revealed that one repeat is dispensable for virus replication, suggesting that construction of such a deleted virus is also possible for related herpesviruses with a genome that can assume one of two isomeric conformations. In addition, such a deletion mutant may be employed to accommodate the insertion and expression of foreign gene(s) that total to at least 13 kbp. The findings that the vL11ΔIR showed reduced plaque size and delayed growth in RK13 cells clearly suggest that the deletion of sequences including the genes within the IR affects the biological properties of EHV-1 in cell culture.

### Example 4

### Protein Expression of the IE and Representative Early and Late Genes was Delayed in vL11ΔIR-Infected Cells

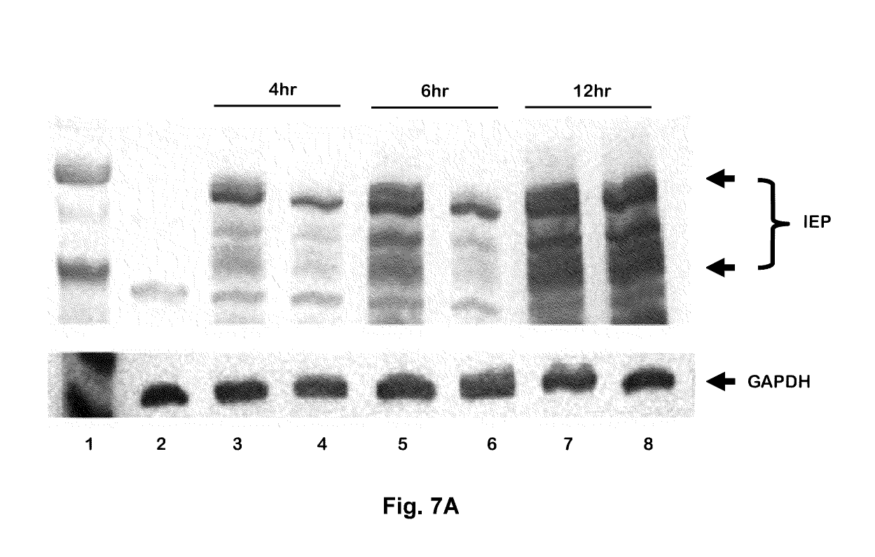

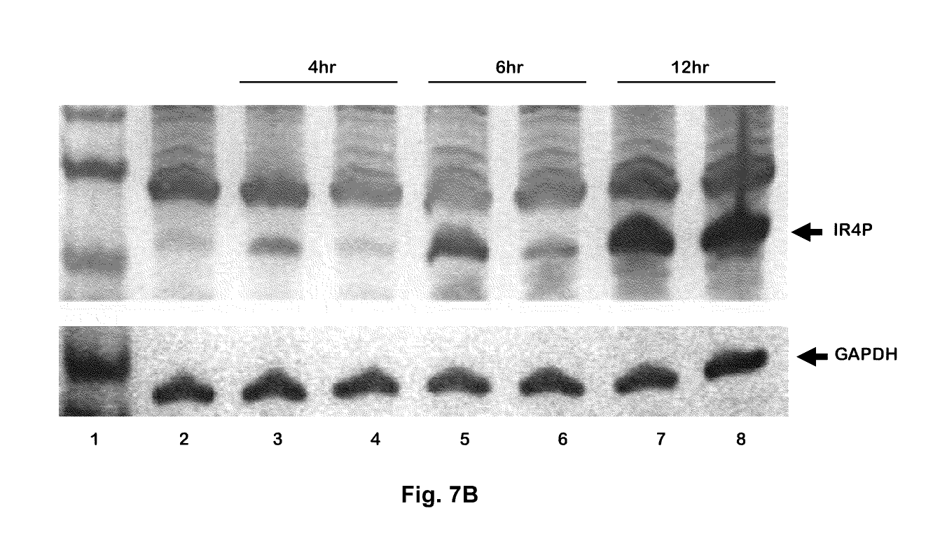

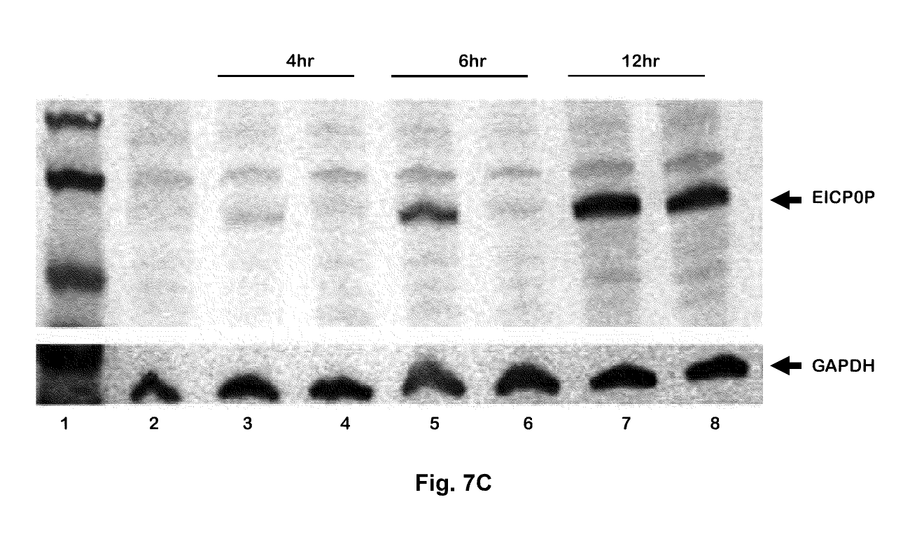

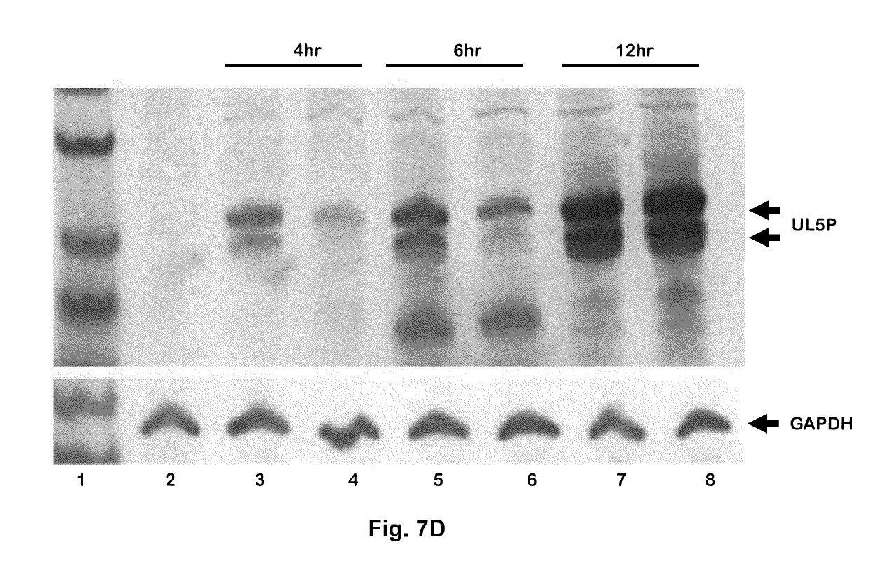

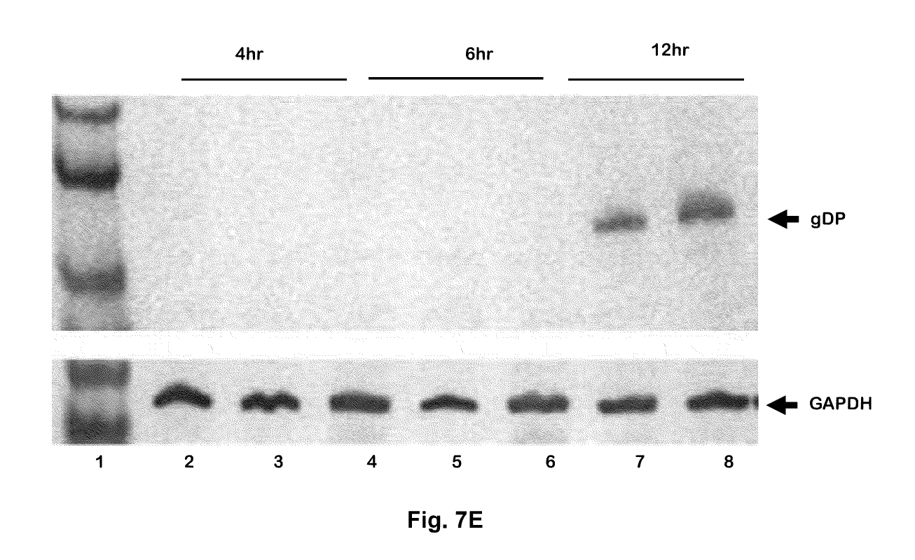

The change of phenotype and the delayed growth kinetics of vL11ΔIR suggested that the deletion of the IR may affect viral gene regulation such that proteins encoded by IR genes would be decreased in cells infected with the IR deleted virus. Therefore, the protein expression of the IR and representative early (IR4, EICP0, and UL5), and late (gD) genes were compared from cells infected with either wild type EHV-1 or the IR deleted virus. FIGS. 7A-7E show the comparison of the expression of viral immediate-early, early, and late proteins in RK13 cells infected with RacL11 EHV-1 or vL11ΔIR as detected by Western blot analyses. For these figures, the lane assignments are as follows: lane 1: protein markers; lane 2: mock-infected RK13 cells; lanes 3, 5 and 7: RacL11-infected RK13 cells; and lanes 4, 6 and 8: vL11ΔIR-infected RK13 cells. GAPDH was used to normalize protein loading. RK13 cells were infected with RacL11 EHV-1 or vL11ΔIR at a moi of 5, whole cell lysates were prepared, and viral proteins were detected as described in Example 1.

FIG. 7A shows the detection for the IE protein. The IE protein (IEP) was detected at 4 hpi in cells infected with either virus (FIG. 7A, lanes 3 and 4), but the amount of the IEP was significantly greater in cells infected with parental RacL11 EHV-1 until 6 hpi (FIG. 7A, lanes 5 and 6). However, by late times of infection, the amount of the IEP was similar in cells infected with either virus (FIG. 7A, lanes 7 and 8).

FIGS. 7B, 7C and 7D show the results for the early gene products, IR4P, EICPOP, and U_{L}5P, respectively. In the case of the EHV-1 early gene products, a similar pattern was observed at early times after infection (4 and 6 hpi), and there was reduced synthesis of the early viral proteins in cells infected with the IR deleted virus. On the other hand, by late times (12 hpi), the amounts of early proteins were similar in cells infected with either the parental or the IR-deleted virus. This pattern of delayed early protein synthesis is shown for the early regulatory proteins IR4P (FIG. 7B), EICPOP (FIG. 7C), and UL5P (FIG. 7D). Lastly, the synthesis of a late EHV-1 gene product, glycoprotein D, was also reduced in cells infected with the vL11ΔIR when compared to cells infected with parental virus. These results are shown in FIG. 7E). Therefore, these results shown in FIGS. 7A-7E indicate that there was an overall delay in EHV-1 protein synthesis in cells infected with a virus mutant that harbored only one copy of the short region 12.7 kbp repeat sequence.

### Example 5

### vL11ΔIR EHV-1 Exhibited Decreased Virulence in CBA Mice

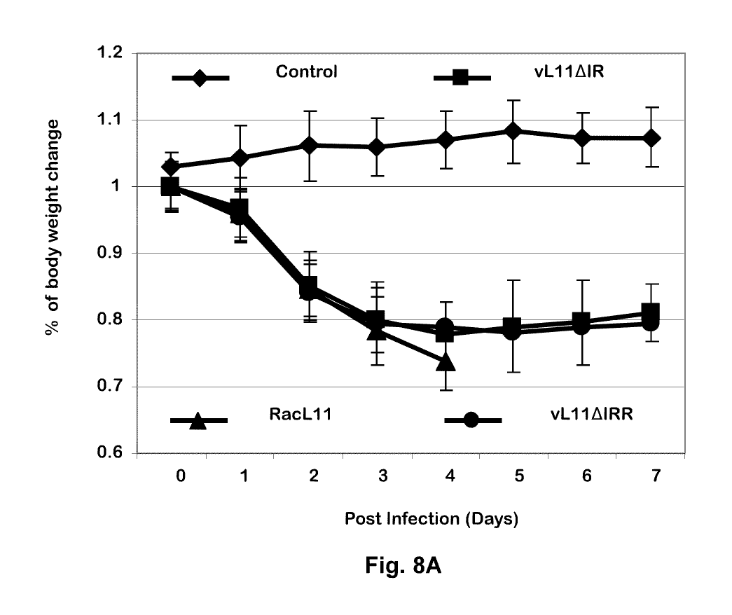

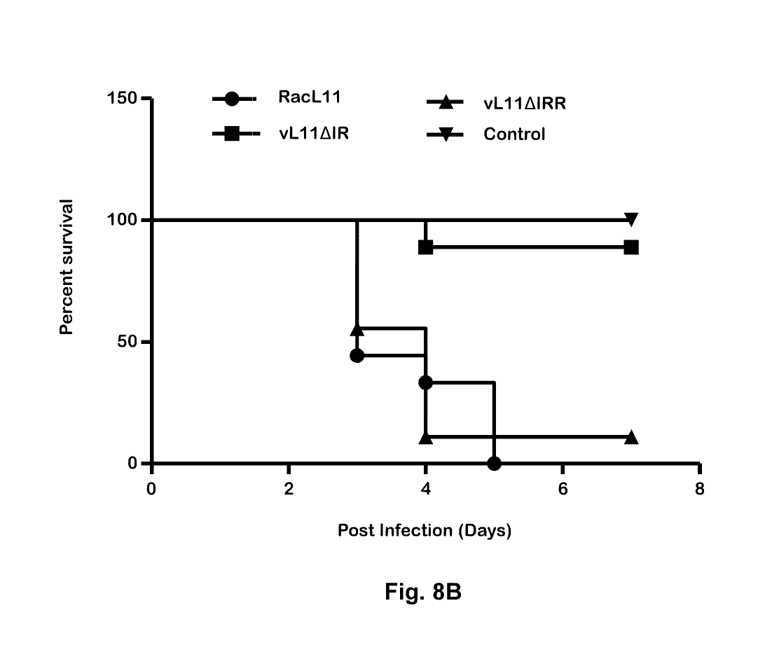

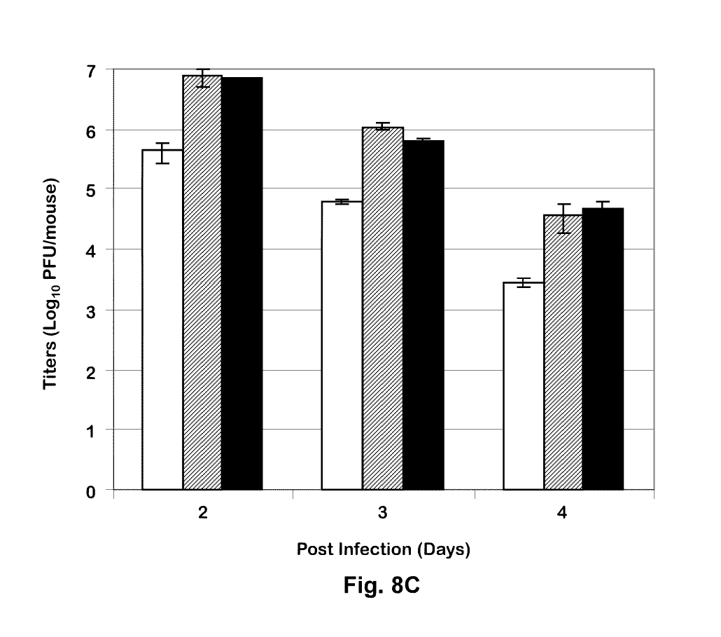

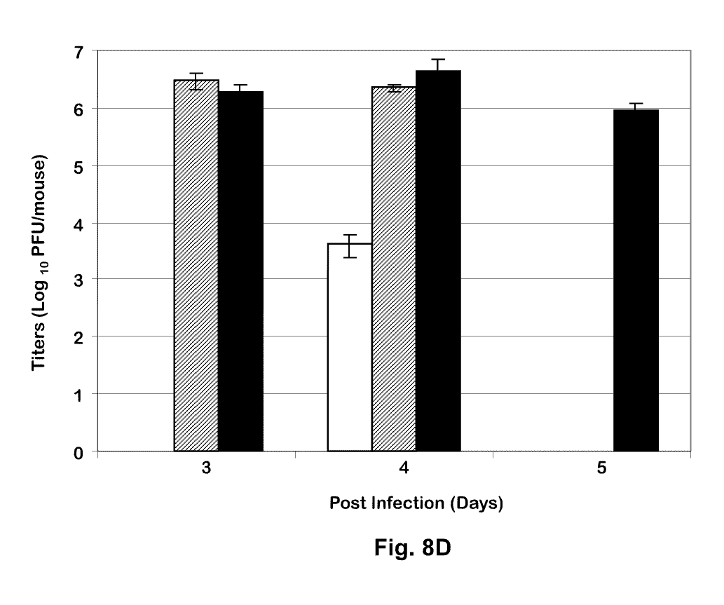

Experiments were carried out to determine if the deletion of the IR affected virulence in the well-characterized CBA mouse model of EHV-1 pathogenesis (Frampton et al., 2002; O'Callaghan and Osterrieder, 2008; Osterrieder et al., 1996b; Smith et al., 2005; von Einem et al., 2004). FIGS. 8A-8D show the percentage change in body weight and percent survival of mock infected mice and mice infected with RacL11, vL11ΔIR, or vL11ΔIRR, and EHV-1 titers of mouse lungs. CBA mice were intranasally inoculated with sterile medium as control or infected with 1×10⁶ PFU of RacL11 EHV-1, vL11ΔIR, or vL11ΔIRR, and total virus was isolated from mouse lungs as described in Example 1. Body weight was measured daily, and the Student's-t test was used to compare measurements of body weight between groups. Error bars indicate standard deviations. FIG. 8A shows the percentage change in body weight of control CBA mice (n=5) or mice infected with RacL11 (n=9), vL11ΔIR (n=9), or vL11ΔIRR (n=9). FIG. 8B shows the percent survival of mock infected mice (n=5), and mice infected with RacL11 EHV-1 (n=9), vL11ΔIR (n=9), or vL11ΔIRR (n=9). FIG. 8C shows the viral titers from lungs of live mice infected with RacL11 EHV-1 (n=3, black bars), vL11ΔIR (n=3, empty bars), or vL11ΔIRR (n=3, cross-hatched bars) at days 2, 3, and 4 post infection. FIG. 8D shows viral titers of lungs from mice that succumbed to infection with RacL11 (black bars), vL11ΔIR (empty bars), or vL11ΔIRR (cross-hatched bars). In FIG. 8D, the number of mice that succumbed at each day during 3 dpi to 5 dpi are n=4 (bar 1), n=5 (bar 2), n=1 (bar 3), n=4 (bar 4), n=1 (bar 5), and n=3 (bar 6)

CBA mice infected intranasally with RacL11, vL11ΔIR, or vL11ΔIRR showed clinical signs of huddling, ruffled fur, lethargy, and significant loss of body weight from 2 dpi, whereas mock-infected control mice continued to gain weight and showed no clinical signs throughout the observation period (FIG. 8A). Mice infected with RacL11, vL11ΔIR, or vL11ΔIRR lost 20% or more of their total body weight by 3 dpi. An overall comparison of body weight loss of the three mouse groups infected with RacL11, vL11ΔIR, or vL11ΔIRR showed there was no significant difference (p>0.8).

Mortality was observed in all groups of mice, and 100% (9 of 9), 11% (1 of 9), and 89% (8 of 9) of mice infected with parental EHV-1, IR-deleted virus, and IR-restored virus, respectively, succumbed to infection. Differences in the virulence among RacL11, vL11ΔIR, and vL11ΔIRR were examined by monitoring the percent of survival as shown in FIG. 8B. Survival curve comparisons showed that survival, following infection with the RacL11 (or vL11ΔIRR) and vL11ΔIR, was significantly different (p>0.008), indicating that the deletion of the IR led to decreased virulence of EHV-1 in this animal model. Lung virus titers of mice necropsied at various days post infection were approximately 10-fold higher in the case of mice infected with the parental virus and vL11ΔIRR virus when compared to those of animals infected with the vL11ΔIR (FIG. 8C). Similarly, high virus titers were seen in the case of lungs of mice that had succumbed to infection with wild type EHV-1 and IR-restored EHV-1 (FIG. 8D). Overall, the animal studies revealed that absence of the IR sequence attenuated EHV-1 virulence in the mouse, and also reduced the ability of the mutant virus to replicate in the lung to high titers.

The finding that the vL11ΔIR was less virulent than the parental virus as judged by overall mortality was attributed to the inability of this ΔIR mutant to replicate to high titers in the murine lung. Whereas the EHV-1 mutant virus lacking both copies of the IR4 gene was completely avirulent, we showed above that the ΔIR virus that harbors and expresses one copy of the IR4 gene and one copy of the IR6 gene, a known determinant of virulence (Osterrieder et al., 1996b), could replicate in the mouse respiratory system and elicit a fatal outcome in a small percentage of the animals.

### REFERENCES

Ahn, B. C., Breitenbach, J. E., Kim, S. K., O'Callaghan, D. J. (2007). The equine herpesvirus-1 IR3 gene that lies antisense to the sole immediate-early (1E) gene is trans-activated by the IE protein, and is poorly expressed to a protein. Virology 363(1), 15-25.
Ahn, B. C., Zhang, Y., O'Callaghan, D. J. (2010). The equine herpesvirus-1 (EHV-1) IR3 transcript downregulates expression of the IE gene and the absence of IR3 gene expression alters EHV-1 biological properties and virulence. Virology in press.
Boldogkoi, Z., Braun, A., Medveczky, I., Glavits, R., Gyuro, B., Fodor, I. (1998). Analysis of the equalization of inverted repeats and neurovirulence using a pseudorabies virus mutant strain altered at the Ul/Ir junction. Virus Genes 17(1), 89-98.
Bowles, D. E., Holden, V. R., Zhao, Y., O'Callaghan, D. J. (1997). The ICPO protein of equine herpesvirus 1 is an early protein that independently transactivates expression of all classes of viral promoters. J Virol 71(7), 4904-14.
Breeden, C. A., Yalamanchili, R. R., Colle, C. F., O'Callaghan, D. J. (1992). Identification and transcriptional mapping of genes encoded at the IR/Us junction of equine herpesvirus type 1. Virology 191(2), 649-60.
Breitenbach, J. E., Ebner, P. D., O'Callaghan, D. J. (2009). The IR4 auxiliary regulatory protein expands the in vitro host range of equine herpesvirus 1 and is essential for pathogenesis in the murine model. Virology 383(2), 188-94.
Caughman, G. B., Lewis, J. B., Smith, R. H., Harty, R. N., O'Callaghan, D. J. (1995). Detection and intracellular localization of equine herpesvirus 1 IR1 and IR2 gene products by using monoclonal antibodies. J Virol 69(5), 3024-32.
Caughman, G. B., Staczek, J., O'Callaghan, D. J. (1985). Equine herpesvirus type 1 infected cell polypeptides: evidence for immediate early/early/late regulation of viral gene expression. Virology 145(1), 49-61.
Chen, M., Garko-Buczynski, K. A., Zhang, Y., O'Callaghan, D. J. (1999). The defective interfering particles of equine herpesvirus 1 encode an ICP22/ICP27 hybrid protein that alters viral gene regulation. Virus Res 59(2), 149-64.
Chen, M., Harty, R. N., Zhao, Y., Holden, V. R., O'Callaghan, D. J. (1996). Expression of an equine herpesvirus 1 ICP22/ICP27 hybrid protein encoded by defective interfering particles associated with persistent infection. J Virol 70(1), 313-20.
Ebner, P. D., Kim, S. K., O'Callaghan, D. J. (2008). Biological and genotypic properties of defective interfering particles of equine herpesvirus 1 that mediate persistent infection. Virology 381(1), 98-105.
Ebner, P. D., O'Callaghan, D. J. (2006). Genetic complexity of EHV-1 defective interfering particles and identification of novel IR4/UL5 hybrid proteins produced during persistent infection. Virus Genes 32(3), 313-20.
Flowers, C. C., O'Callaghan, D. J. (1992). Equine herpesvirus 1 glycoprotein D: mapping of the transcript and a neutralization epitope. J Virol 66(11), 6451-60.
Frampton, A. R., Jr., Smith, P. M., Zhang, Y., Matsumura, T., Osterrieder, N., O'Callaghan, D. J. (2002). Contribution of gene products encoded within the unique short segment of equine herpesvirus 1 to virulence in a murine model. Virus Res 90(1-2), 287-301.
Garko-Buczynski, K. A., Smith, R. H., Kim, S. K., O'Callaghan, D. J. (1998). Complementation of a replication-defective mutant of equine herpesvirus type 1 by a cell line expressing the immediate-early protein. Virology 248(1), 83-94.
Grundy, F. J., Baumann, R. P., O'Callaghan, D. J. (1989). DNA sequence and comparative analyses of the equine herpesvirus type 1 immediate early gene. Virology 172(1), 223-36.
Guggemoos, S., Just, F. T., Neubauer, A. (2006). The equine herpesvirus 1 UL20 product interacts with glycoprotein K and promotes egress of mature particles. J Virol 80(1), 95-107.
Harty, R. N., Colle, C. F., O'Callaghan, D. J. (1990). Equine herpesvirus type 1 gene regulation: Characterization of transcription from the immediate early gene in productive infection. In “Herpesvirus Transcriptional and Its Regulation” (E. K. Wagner, Ed.), pp. 319-338. CRC Press, Inc., Boca Raton, Fla.
Henry, B. E., Robinson, R. A., Dauenhauer, S. A., Atherton, S. S., Hayward, G. S., O'Callaghan, D. J. (1981). Structure of the genome of equine herpesvirus type 1. Virology 115(1), 97-114.
Holden, V. R., Caughman, G. B., Zhao, Y., Harty, R. N., O'Callaghan, D. J. (1994). Identification and characterization of the ICP22 protein of equine herpesvirus 1. J Virol 68(7), 4329-40.
Holden, V. R., Harty, R. N., Yalamanchili, R. R., O'Callaghan, D. J. (1992a). The IR3 gene of equine herpesvirus type 1: a unique gene regulated by sequences within the intron of the immediate-early gene. DNA Seq 3(3), 143-52.
Holden, V. R., Yalamanchili, R. R., Harty, R. N., O'Callaghan, D. J. (1992b). Identification and characterization of an equine herpesvirus 1 late gene encoding a potential zinc finger. Virology 188(2), 704-13.
Holden, V. R., Zhao, Y., Thompson, Y., Caughman, G. B., Smith, R. H., O'Callaghan, D. J. (1995). Characterization of the regulatory function of the ICP22 protein of equine herpesvirus type 1. Virology 210(2), 273-82.
Jenkins, F. J., Donoghue, A. M., Martin, J. R. (1996). Deletion of the Herpes simplex 1 internal repeat sequences affects pathogenicity in the mouse. Front Biosci 1, a59-68.
Jenkins, F. J., Martin, J. R. (1990). Role of the herpes simplex virus 1 internal repeat sequences in pathogenicity. Intervirology 31(2-4), 129-38.
Jenkins, F. J., Roizman, B. (1986). Herpes simplex virus 1 recombinants with non-inverting genomes frozen in different isomeric arrangements are capable of independent replication. J Virol 59(2), 494-9.
Kim, S. K., Ahn, B. C., Albrecht, R. A., O'Callaghan, D. J. (2006). The unique IR2 protein of equine herpesvirus 1 negatively regulates viral gene expression. J Virol 80(10), 5041-9.
Neubauer, A., Osterrieder, N. (2004). Equine herpesvirus type 1 (EHV-1) glycoprotein K is required for efficient cell-to-cell spread and virus egress. Virology 329(1), 18-32.
O'Callaghan, D. J., Colle, C. F., 3rd, Flowers, C. C., Smith, R. H., Benoit, J. N., Bigger, C. A. (1994). Identification and initial characterization of the IR6 protein of equine herpesvirus 1. J Virol 68(9), 5351-64.
O'Callaghan, D. J., Osterrieder, N. (2008). Herpesviruses of Horses. In “Encyclopedia of Virology” (B. W. J. M. a. M. H. V. VanRegenmortel, Ed.), pp. 411-420. Elsevier Ltd, Oxford.
Osterrieder, N., Neubauer, A., Brandmuller, C., Braun, B., Kaaden, O. R., Baines, J. D. (1996a). The equine herpesvirus 1 glycoprotein gp21/22a, the herpes simplex virus type 1 gM homolog, is involved in virus penetration and cell-to-cell spread of virions. J Virol 70(6), 4110-5.
Osterrieder, N., Neubauer, A., Brandmuller, C., Kaaden, O. R., O'Callaghan, D. J. (1996b). The equine herpesvirus 1 IR6 protein influences virus growth at elevated temperature and is a major determinant of virulence. Virology 226(2), 243-51.
Osterrieder, N., Neubauer, A., Brandmuller, C., Kaaden, O. R., O'Callaghan, D. J. (1998). The equine herpesvirus 1 IR6 protein that co-localizes with nuclear lamins is involved in nucleocapsid egress and migrates from cell to cell independently of virus infection. J Virol 72(12), 9806-17.
Perdue, M. L., Kemp, M. C., Randall, C. C., O'Callaghan, D. J. (1974). Studies of the molecular anatomy of the L-M cell strain of equine herpes virus type 1: proteins of the nucleocapsid and intact virion. Virology 59(1), 201-16.
Poffenberger, K. L., Roizman, B. (1985). A non-inverting genome of a viable herpes simplex virus 1: presence of head-to-tail linkages in packaged genomes and requirements for circularization after infection. J Virol 53(2), 587-95.
Poffenberger, K. L., Tabares, E., Roizman, B. (1983). Characterization of a viable, non-inverting herpes simplex virus 1 genome derived by insertion and deletion of sequences at the junction of components L and S. Proc Natl Acad Sci USA 80(9), 2690-4.
Roizman, B. (1996). Herpesviridae. third ed. In “Field Virology” (B. N. Field, Knipe, D. M., Howley, P. M., Chanock, R. M., Melnick, J. L., Nonath, R. P., Roizman, B., Straus, S. E., Ed.), pp. 2221-2230. Lippincott Williams & Wilkins, Philadelphia, Pa.
Roizman, B., Pellet, P. E. (2001). The Family of Herpesviridae: a brief introduction. Fourth ed. In “Field Virology” (D. M. Knipe, and P. M. Howley, Eds.), pp. 2381-2397. Lippincott Williams and Wilkins., Philadelphia, Pa.
Rosas, C., Van de Walle, G. R., Metzger, S. M., Hoelzer, K., Dubovi, E. J., Kim, S. G., Parrish, C. R., Osterrieder, N. (2008). Evaluation of a vectored equine herpesvirus type 1 (EHV-1) vaccine expressing H3 haemagglutinin in the protection of dogs against canine influenza. Vaccine 26(19), 2335-43.
Rudolph, J., O'Callaghan, D. J., Osterrieder, N. (2002). Cloning of the genomes of equine herpesvirus type 1 (EHV-1) strains KyA and racL11 as bacterial artificial chromosomes (BAC). J Vet Med B Infect Dis Vet Public Health 49(1), 31-6.
Ruyechan, W. T., Dauenhauer, S. A., O'Callaghan, D. J. (1982). Electron microscopic study of equine herpesvirus type 1 DNA. J Virol 42(1), 297-300.
Sauer, A., Wang, J. B., Hahn, G., McVoy, M. A. (2010). A human cytomegalovirus deleted of internal repeats replicates with near wild type efficiency but fails to undergo genome isomerization. Virology 401(1), 90-5.
Smith, P. M., Kahan, S. M., Rorex, C. B., von Einem, J., Osterrieder, N., O'Callaghan, D. J. (2005). Expression of the full-length form of gp2 of equine herpesvirus 1 (EHV-1) completely restores respiratory virulence to the attenuated EHV-1 strain KyA in CBA mice. J Virol 79(8), 5105-15.
Smith, R. H., Caughman, G. B., O'Callaghan, D. J. (1992). Characterization of the regulatory functions of the equine herpesvirus 1 immediate-early gene product. J Virol 66(2), 936-45.
Smith, R. H., Zhao, Y., O'Callaghan, D. J. (1993). The equine herpesvirus 1 (EHV-1) UL3 gene, an ICP27 homolog, is necessary for full activation of gene expression directed by an EHV-1 late promoter. J Virol 67(2), 1105-9.
Srinivasan, R., Fink, D. J., Glorioso, J. C. (2008). HSV vectors for gene therapy of chronic pain. Curr Opin Mol Ther 10(5), 449-55.
Telford, E. A., Watson, M. S., McBride, K., Davison, A. J. (1992). The DNA sequence of equine herpesvirus-1. Virology 189(1), 304-16.

Tischer, B. K., von Einem, J., Kaufer, B., Osterrieder, N. (2006). Two-step red-mediated recombination for versatile high-efficiency markerless DNA manipulation in Escherichia coli. Biotechniques 40(2), 191-7.
Trapp, S., von Einem, J., Hofmann, H., Kostler, J., Wild, J., Wagner, R., Beer, M., Osterrieder, N. (2005). Potential of equine herpesvirus 1 as a vector for immunization. J Virol 79(9), 5445-54.
von Einem, J., Wellington, J., Whalley, J. M., Osterrieder, K., O'Callaghan, D. J., Osterrieder, N. (2004). The truncated form of glycoprotein gp2 of equine herpesvirus 1 (EHV-1) vaccine strain KyA is not functionally equivalent to full-length gp2 encoded by EHV-1 wild-type strain RacL11. J Virol 78(6), 3003-13.
Warming, S., Costantino, N., Court, D. L., Jenkins, N. A., Copeland, N. G. (2005). Simple and highly efficient BAC recombineering using GalK selection. Nucleic Acids Res 33(4), e36.
Whalley, J. M., Robertson, G. R., Davison, A. J. (1981). Analysis of the genome of equine herpesvirus type 1: arrangement of cleavage sites for restriction endonucleases EcoRI, BglII and BamHI. J Gen Virol 57(Pt 2), 307-23.
Whalley, J. M., Ruitenberg, K. M., Sullivan, K., Seshadri, L., Hansen, K., Birch, D., Gilkerson, J. R., Wellington, J. E. (2007). Host cell tropism of equine herpesviruses: glycoprotein D of EHV-1 enables EHV-4 to infect a non-permissive cell line. Arch Virol 152(4), 717-25.
Yamada, H., Daikoku, T., Yamashita, Y., Jiang, Y. M., Tsurumi, T., Nishiyama, Y. (1997). The product of the US10 gene of herpes simplex virus type 1 is a capsid/tegument-associated phosphoprotein which co-purifies with the nuclear matrix. J Gen Virol 78 (Pt 11), 2923-31.
Yokoyama, H., Sasaki, K., Franks, M. E., Goins, W. F., Goss, J. R., Degroat, W. C., Glorioso, J., Chancellor, M. B., Yoshimura, N. (2008). Gene Therapy for Bladder Overactivity and Nociception with Herpes Simplex Virus Vectors Expressing Preproenkephalin. Hum Gene Ther.
Zhao, Y., Holden, V. R., Smith, R. H., O'Callaghan, D. J. (1995). Regulatory function of the equine herpesvirus 1 ICP27 gene product. J Virol 69(5), 2786-93.

The complete disclosures of all references cited in this application are hereby incorporated by reference. Specifically incorporated into this application are the following two documents: (1) Provisional application Ser. No. 61/521,131 filed Aug. 8, 2011; and (2) B. Ahn, Y. Zhung, N. Osterrieder, and D. J. O'Callaghan; “Properties of an equine herpesvirus 1 mutant devoid of the internal inverted repeat sequence of the genomic short region,” Virology, vol. 410, pp. 327-335 (2011), available online 21 Dec. 2010. In the event of an otherwise irreconcilable conflict, however, the present specification shall control.

What is claimed:

1. A mutant, replicating equine herpesvirus-1 in which the genome lacks the coding sequence for one internal repeat segment.

2. The mutant equine herpesvirus-1 as in claim 1, whose genome lacks a single copy of the genes for IR1, IR2, IR3, IR4, IR5, IR6, and a portion of Us1 gene.

3. The mutant equine herpesvirus-1 as in claim 1, whose genome lacks about 12,716 base nucleotide base pairs from about 112,933 to about 125,649 base pairs on one chromosome.

4. The mutant equine herpesvirus-1 as in claim 1, wherein the genome further comprises a nucleotide sequence encoding one or more exogenous genes selected from the group consisting of genes encoding immunomodulatory proteins and genes encoding antigens for infectious diseases other than EHV-1.

5. The mutant equine herpesvirus-1 as in claim 4, wherein the exogenous gene additionally comprises one or more exogenous promoter genes that control expression of the one or more exogenous genes.

6. The mutant equine herpesvirus-1 as in claim 5, wherein the one or more promoter genes are selected from the group consisting of the EHV-1 immediate-early gene promoter, the EHV-1 tk gene promoter, the EHV-1 gp13 gene promoter, the EHV-1 gp 14 gene promoter, and the human cytomegalovirus immediate early gene promoter.

7. The mutant equine herpesvirus-1 as in claim 4, wherein one or more immunomodulatoy proteins are selected from the group consisting of interferon, interleukin-12, and interleukin-2.

8. The mutant equine herpesvirus-1 as in claim 4, wherein one or more antigens of additional diseases are selected from the group consisting of Rabies G protein, equine infectious anemia ENV protein gp70-gp45, Eastern equine encephalitis virus E1 membrane protein, Eastern equine encephalitis virus E2 membrane protein, Venezuelan equine encephalitis virus E1 membrane protein, Venezuelan equine encephalitis virus E2 membrane protein, equine influenza virus hemagglutinin, equine influenza virus H3 protein, equine arteritis virus G1 membrane protein, equine arteritis virus G2 membrane protein, yellow fever virus prm-E protein, equine herpesvirus-4 glycoprotein gD, and other equine herpesviruses glycoproteins.

9. A composition comprising the mutant equine herpesvirus of claim 1 and a pharmaceutically acceptable vehicle.

10. A vaccine for equine herpesvirus 1 comprising the mutant equine herpesvirus of claim 1 and a pharmaceutically acceptable carrier.

11. The vaccine of claim 10, further comprising one or more adjuvants.

12. The vaccine of claim 11, wherein the one or more adjuvants are selected from the group consisting of cross-linked polymers, complete Freund's adjuvant, incomplete Freund's adjuvant, aluminum hydroxide, dimethyldioctadecylammonium bromide, Adjuvax, Imject Alum, Monophosphoryl Lipid A, Titermax, vitamin E acetate solubilisate, aluminum phosphate, aluminum oxide, squalene, glycolipids, glycoproteins, lipids, oils, bacterial cell walls, and saponins.

13. A method to decrease the symptoms of a horse from exposure to wildtype equine herpesvirus 1, said method comprising administering to the horse an immunologically effective amount of the vaccine of claim 10 sufficiently prior to such exposure to generate an immune response in the horse.

14. A method of immunizing a horse against infection by equine herpesvirus-1, said method comprising administering to the horse the vaccine of claim 10.

15. A viral vector comprising the mutant equine herpesvirus-1 of claim 1.

16. The viral vector of claim 15, wherein said viral vector can replicate in mammals.

17. The viral vector of claim 16, wherein said mammals are selected from the group consisting of mice, horses, rabbits, monkeys, and humans.


SEQUENCE LISTING (20 sequences)

SEQ ID NO: 1 - DNA, 78 bp, Artificial sequence
  Synthetic primer
ccgggccatatctggtcaagggtcacgggcccgcgcccgagagagagcctggcccctgttgacaattaatcatcggca

SEQ ID NO: 2 - DNA, 72 bp, Artificial sequence
  Synthetic primer
acaccgtagtgggtgagtgtgggttttccaaacatagctcgaattcattagttcagcactgtcctgctcctt

SEQ ID NO: 3 - DNA, 30 bp, Artificial sequence
  Synthetic primer
atgatcccgcagttacagcctacaaactgg

SEQ ID NO: 4 - DNA, 29 bp, Artificial sequence
  Synthetic primer
tagcacacctaacctcctgagtgtgagcg

SEQ ID NO: 5 - DNA, 31 bp, Artificial sequence
  Synthetic primer
agttgatggataggcgagcatctcaaacaag

SEQ ID NO: 6 - DNA, 34 bp, Artificial sequence
  Synthetic primer
tgaaacatctgcaactgcgtaacaacagcttcgg

SEQ ID NO: 7 - DNA, 32 bp, Artificial sequence
  Synthetic primer
agatgtatatctgccaggctctctctcgggcg

SEQ ID NO: 8 - DNA, 37 bp, Artificial sequence
  Synthetic primer
agatatacatctactaatgaattcgagctatgtttgg

SEQ ID NO: 9 - DNA, 30 bp, Artificial sequence
  Synthetic primer
ttctctttggatggtataagacaatcgtcg

SEQ ID NO: 10 - DNA, 32 bp, Artificial sequence
  Synthetic primer
acacattgagtcctttctactctcctcctcgg

SEQ ID NO: 11 - DNA, 22 bp, Artificial sequence
  Synthetic primer
ccgtttgaatgcgattggtggg

SEQ ID NO: 12 - DNA, 22 bp, Artificial sequence
  Synthetic primer
gcgttgtatctagcagcccacg

SEQ ID NO: 13 - DNA, 21 bp, Artificial sequence
  Synthetic primer
agagtaggcgttccatccacg

SEQ ID NO: 14 - DNA, 22 bp, Artificial sequence
  Synthetic primer
gaccctaccaaaggcgtgtagg

SEQ ID NO: 15 - DNA, 27 bp, Artificial sequence
  Synthetic primer
cctgttgacaattaatcatcggcatag

SEQ ID NO: 16 - DNA, 22 bp, Artificial sequence
  Synthetic primer
actgtcctgctccttgtgatgg

SEQ ID NO: 17 - DNA, 38 bp, Artificial sequence
  Synthetic primer
tttgaattcgccaccatgggggcctgctgctcctctag

SEQ ID NO: 18 - DNA, 34 bp, Artificial sequence
  Synthetic primer
ttatgtacaattcagaccgtatatggtgttttgc

SEQ ID NO: 19 - DNA, 24 bp, Artificial sequence
  Synthetic primer
catgtttgtgatgggcgtgaacca

SEQ ID NO: 20 - DNA, 22 bp, Artificial sequence
  Synthetic primer
taagcagttggtggtgcaggat